(19) United States
(12) Patent Application Publication
(10) Pub. No.: US 2012/0171734 A1
(43) Pub. Date: Jul. 5, 2012

(54) EXTRACTION OF EXTRACELLULAR TERPENOIDS FROM MICROALGAE COLONIES

(75) Inventors: Anastasios Melis, El Cerrito, CA (US); Ela Eroglu, Berkley, CA (US)

(73) Assignee: THE REGENTS OF THE UNIVERSITY OF CALIFORNIA, Oakland, CA (US)

(21) Appl. No.: 13/381,474

(22) PCT Filed: Jul. 1, 2010
(86) PCT No.: PCT/US10/40824

§ 371 Date: Mar. 23, 2012

Related U.S. Application Data
(60) Provisional application No. 61/222,410, filed on Jul. 1, 2009.

(51) Int. Cl. C12P 17/04; C12P 5/00

(52) U.S. Cl. 435/126; 435/167

(57) ABSTRACT

The invention provides methods of extracting and quantifying extracellular terpenoid hydrocarbons, e.g., botryococcenes, methylated squalenes, and carotenoids, from terpenoid-producing and secreting green microalgae.

### CROSS-REFERENCE TO RELATED APPLICATIONS

This application claims benefit of U.S. provisional patent application No. 61/222,410, filed Jul. 1, 2009, which is herein incorporated by reference.

### BACKGROUND OF THE INVENTION

A variety of hydrocarbon-accumulating microalgae exist. These include members of the genus Botryococcus. This genus encompasses a variety of hydrocarbon-accumulating green microalgae that are classified in three major races on the basis of the chemical structure of the hydrocarbons produced. Race A produces odd-numbered (C₂₃-C₃₃) n-alkadienes (mainly diene and triene hydrocarbons), race B produces triterpenoid hydrocarbons such as C₃₀-C₃₇ botryococcenes and C₃₁-C₃₄ methylated squalenes, whereas race L produce lycopadienes, which are single tetraterpenoid hydrocarbons (Metzger and Largeau, Appl. Microbiol. Biotechnol. 66:486-496, 2005). The B-race represents a group of microcolony-forming green microalgae with individual cell sizes of about 10 μm in length. These microalgae synthesize long-chain terpenoid hydrocarbons via the plastidic DXP-MEP pathway (Lichtenthaler, Ann. Rev. Plant. Physiol. Plant. Mol. Biol. 50:47-65, 1999; Koppisch et al., Organic. Lett. 2:215-217, 2000) and deposit them in the extracellular space, thus forming a hydrophobic matrix to which multiple individual cells adhere (Banerjee et al., Crit. Rev. Biotechnol. 22:245-279, 2002; Sato et al., Tetrahedron Lett. 44:7035-7037, 2003; Metzger and Largeau, supra, 2005). Botryococcene hydrocarbons are modified triterpenes, having the chemical formula CₙH₂ₙ₋₁₀ (Banerjee et al., supra 2002). Botryococcene hydrocarbons, produced by the B race, can accumulate up to 30-40% of the dry biomass weight (Metzger and Largeau, supra, 2005). The high level of botryococcene hydrocarbons and the ability of these colonial microalgae to form blooms have raised the prospect of their commercial exploitation for the production of synthetic chemistry and biofuel feedstocks (Casadevall et al., Biotechnol. Bioeng. 27:286-295, 1985). It was suggested that C₃₀-C₃₇ botryococcenes and C₃₁-C₃₄ methylated squalenes could be converted via catalytic cracking into shorter-length fuel-type hydrocarbons, such as C₇Hₙ through C₁₁Hₘ for gasoline, C₁₂-C₁₅ for kerosene (jet fuel), or C₁₆-C₁₈ for diesel, (Hillen et al., Biotechnol. Bioeng. 24:193-205, 1982). Interestingly, geochemical analysis of petroleum has shown that botryococcene-type hydrocarbons, presumably generated by microalgae ancestral to Botryococcus braunii, may be the source of today's petroleum deposits (Moldowan and Seifert, JCS Chem. Comm. 19:912-914, 1980). Accordingly, botryococcene hydrocarbon production by photosynthetic CO₂ fixation in microalgae may provide a source of renewable fuel, mitigate emission of greenhouse gases in the atmosphere, and prevent climate change (Metzger and Largeau, supra, 2005).

Colonies of B. braunii typically have amorphous structures, with a morphology characterized by a “botryoid” organization of individual pyriform-shaped cells, held together by a thick hydrocarbon matrix. It has been reported that the matrix surrounding individual cells forms an outer cell wall and that the bulk of B. braunii hydrocarbons are stored in these extracellular containment structures (Largeau et al., Phytochem. 19:1043-1051, 1980). Botryococcene hydrocarbons are also found sequestered within the cells, where the biosynthesis and initial segregation of these molecules take place. Intracellular hydrocarbons are only a small fraction of the total micro-colony hydrocarbon content and they are more difficult to isolate compared to the extracellular matrix (Largeau et al., supra, 1980; Wolf et al., J. Phycol. 21:88-396, 1985).

Hydrocarbon recovery can be achieved by extraction of the dry biomass with solvents (Metzger and Largeau, supra, 2005). Supercritical CO₂ extraction has also been employed and the extraction was found to be optimal at a pressure of 30 MPa (Mendes et al., Inorg. Chim. Acta. 356:328-334, 2003). Contact of the wet biomass with non-toxic solvents has also been reported to be a suitable approach for hydrocarbon extraction (Frenz et al., Enzyme Microb. Technol. 11(11), 717-7241989). There is a need, however, for extraction procedures that are simple, inexpensive and that can isolate hydrocarbons on a large scale.

### BRIEF SUMMARY OF THE INVENTION

This invention is based, in part, on the discovery that gentle disruption of microcolonies without substantial cellular lysis and extraction with a solvent such as heptane or hexane can provide the basis for a simple extraction protocol and spectrophotometric determination of the amount of hydrocarbon extracted. Thus, in on aspect, the invention provides a method for the extraction and spectrophotometric quantitation of extracellular terpenoid hydrocarbons, e.g., triterpenoid C₃₀-C₃₇ hydrocarbons (botryococcenes) and methylated squalenes from green microalgae, e.g., Botryococcus sp., such as B. braunii. For the method can comprise vortexing of microalgae micro-colonies, e.g., B. braunii micro-colonies, with glass beads to remove extracellular hydrocarbons from the micro-colony biomass. Density equilibrium or aqueous/solvent (e.g., a solvent such as heptane or hexane) two-phase partition can then typically be employed to separate these extractable hydrocarbons from the biomass. The invention further provides suitable extinction coefficients to quantify the amount of botryococcenes, methylated squalenes and botryoxanthin extracted from Botryococcus, e.g., B. braunii.

The invention thus provides a method of extracting extracellular C₃₀-C₃₇ botryococcenes and C₃₁-C₃₄ methylated squalene terpenoid hydrocarbons from microalgae micro-colonies, the method comprising: providing a sample comprising microalgae micro-colonies; mechanically dispersing the microalgae micro-colonies, wherein the dispersal is performed without substantially breaking open the cells; extracting the terpenoid hydrocarbons using an organic solvent selected from the group consisting of hexane, heptane or octane to obtain a fraction comprising the organic solvent containing the hydrocarbons; and quantifying the terpenoid hydrocarbons present in the organic solvent fraction spectrophotometrically. In preferred embodiments, the terpenoid hydrocarbons are triterpenoids, e.g., C₃₀-C₃₇ botryococcenes and C₃₁-C₃₄ methylated squalenes. In typical embodiments, the organic solvent is heptane.

In some embodiments, the step of quantifying the botryococcene hydrocarbons present in the organic solvent, e.g., heptane, spectrophotometrically comprises using an extinction coefficient of about 90±5 mM⁻¹ cm⁻¹ for the absorbance of the hydrocarbons at 190 nm.

In preferred embodiments, the microalgae is Botryococcus sp., such as Botryococcus braunii. Further, in some embodiments, the Botryococcus braunii is a Botryococcus braunii, var Showa (the Berkeley strain).

In some embodiments, the steps of mechanically dispersing the microalgae micro-colonies and extracting the terpenoid hydrocarbons is performed concurrently. In typical embodiments, such steps comprise vortexing the microalgae micro-colonies in the organic solvent in the presence of glass beads.

In some embodiments, the method of extracting the extracellular terpenoid hydrocarbons comprise a step of heating the microalgae colony sample to about 100° C. prior to mechanically disrupting the micro-colonies. The step of heating is typically performed for about 10 or 15 minutes.

In some embodiments, the step of mechanically disrupting the micro-colonies comprises sonicating the micro-colonies at low power in the organic solvent, e.g., heptane.

The invention also provides a method of extracting triterpenoid C₃₀-C₃₇ botryococcenes and C₃₁-C₃₄ methylated squalenes from Botryococcus microalgae micro-colonies, the method comprising: providing a sample comprising Botryococcus microalgae micro-colonies; heating the sample to about 100° C. for about 15 or about 10 minutes or less; vortexing the Botryococcus micro-colonies in heptane in the presence of glass beads to obtain a fraction comprising heptane containing the hydrocarbons; and quantifying the botryococcene hydrocarbons present in the organic solvent spectrophotometrically using an extinction coefficient of about 90±5 mM⁻¹ cm⁻¹ for the absorbance of the hydrocarbons at 190 nm. In some embodiments, the Botryococcus sp. is Botryococcus braunii.

In a further aspect, the invention provides a method of extracting extracellular C₄₀ carotenoid hydrocarbons, e.g., botryoxanthin hydrocarbons, from microalgae, the method comprising: providing a sample comprising green algae micro-colonies; vortexing the green algae micro-colonies in heptane in the presence of glass beads to obtain a fraction comprising heptane containing the hydrocarbons; quantifying the botryoxanthin hydrocarbons present in the heptane fraction spectrophotometrically at 450 nm using an extinction coefficient of about 165±5 mM⁻¹ cm⁻¹. In typical embodiments, the microalgae is a Botryococcus sp, such as Botryococcus braunii, e.g., a member of the B race of Botryococcus.

### BRIEF DESCRIPTION OF THE DRAWINGS

FIG. 1. FIG. 1a. Absorbance spectrum of a squalene solution in heptane. The single absorbance band occurs in the 200-800 nm region, peaking at 190 nm. FIG. 1b. (Solid circles) Absorbance at 190 nm of squalene in heptane, plotted as a function of squalene concentration. The slope of the straight line defined the specific absorbance coefficient (extinction coefficient) of squalene in heptane at 190 nm, equal to 90±5 mM-1 cm⁻¹. (Open diamonds). Absorbance at 190 nm of botryococcene in heptane measured in three different samples and plotted as a function of botryococcene concentration. The latter was determined gravimetrically upon a subsequent evaporation of the heptane solvent and weighing of the residue (Botryococcene readings: A190=0.38, C=3.6 μM; A190=0.435, C=4.7 μM; A190=0.56, C=5.7 μM).

FIG. 2. FIG. 2a. Absorbance spectrum of a β-carotene solution in heptane. The typical carotenoid absorbance bands occur in the 400-500 nm region, with the prominent absorbance at 450 nm. FIG. 2b. Absorbance at 450 nm of β-carotene in heptane, plotted as a function of β-carotene concentration. The slope of the straight line defined the specific absorbance coefficient (extinction coefficient) of β-carotene in heptane at 450 nm, equal to 165±5 mM-1 cm⁻¹.

FIG. 3. FIG. 3a. B. braunii var. Showa cultures grown in 500 mL of modified Chu-13 medium in conical Fernbach flasks upon orbital shaking. Oil-rich micro-colonies centrifuge to the center of the H₂O-based growth medium upon orbital shaking. FIG. 3b. Microscopic observation of mechanically compressed micro-colonies of B. braunii var. Showa, revealing droplets of botryococcene hydrocarbons exuding from the micro-colonies into the growth medium.

FIG. 4. FIG. 4a. B. braunii var. Showa dry cell weight biomass harvested from a continuous fed culture. Arrows indicate the points in time, i.e., every 48 h, when a fixed fraction (40% of the culture volume) was harvested and replaced by an equal amount of fresh growth medium. The dry cell weight in grams of the harvested biomass per liter culture is plotted as a function of growth time in the continuous culture. FIG. 4b. Cumulative productivity of B. braunii var. Showa cultures from a continuous fed process, as shown in FIG. 5, and according to the experimental details of FIG. 7. The slope of the straight line defined the rate of biomass accumulation, equal to 125 mg dcw L-1 d-1.

FIG. 5. FIG. 5a. Microscopic observation of a dispersed B. braunii var. Showa micro-colony, showing the grape-seed-like green cells and the yellowish-orange botryococcene-carotenoid matrix (Btc). Nile red staining showed the yellowish-orange matrix to be highly fluorescent, consistent with a highly hydrophobic environment in this matrix. FIG. 5b. Sucrose gradient density equilibrium separation of Botryococcus braunii var. Showa cell biomass and terpenoid hydrocarbons. Micro-colonies were mechanically disrupted prior to the sucrose density centrifugation. A discontinuous 10-80% (w/v), sucrose gradient having a concentration increment step of 10% was employed.

FIG. 6. Aqueous-organic phase partition of the botryococcene-carotenoid-containing heptane upper phase (a) from the B. braunii var. Showa biomass lower phase (b). Also shown are the glass beads used for the mechanical disruption of the microcolonies, resting in the bottom of the conical Falcon centrifuge tube (c). Following the vortexing of the 1 g wet packed cell biomass with glass beads in the presence of 10 ml heptane, 10 ml of B. braunii growth medium was added to the mix, causing separation of the aqueous-organic phases.

FIG. 7. Absorbance spectra of the B. braunii var. Showa heptane extract after vortexing of the micro-colonies with glass beads. Two distinct absorbance bands are seen in the (a) UV-C (˜190 nm), attributed to botryococcenes (dilution scale=1:500), and (b) blue (380-520 nm) region of the spectrum, attributed to carotenoid (dilution scale=1:4), respectively.

FIG. 8. FIG. 8a. Amount of botryococcene extracted from B. braunii var. Showa micro-colonies in control samples (circles) and samples incubated at 100° C. for 10 min. FIG. 8b. Amount of carotenoid extracted from B. braunii var. Showa micro-colonies in control samples (circles) and samples incubated at 100° C. for 10 min (triangles), as a function of vortexing time in the presence of heptane and glass beads.

FIG. 9. Structures of botryococcenes and methylated squalenes.

FIG. 10. Botryococcus cells, grown in 500 mL of modified Chu-13 medium in conical Fernbach flasks upon orbital shaking. (a) Botryococcus braunii var. Showa, (b) Botryococcus braunii var. Kawaguchi-1, (c) Botryococcus braunii var. Yamanaka, (d) Botryococcus braunii var. UTEX 2441, (e) Botryococcus braunii var. UTEX LB-572 micro-colonies centrifuging to the center of the H₂O-based growth medium; (f) Botryococcus sudeticus (UTEX 2629) cultures made uniform suspension.

FIG. 11. Cumulative biomass productivities of Botryococcus strains in continuous fed cultures. Data points indicate the time when a fixed fraction of the culture (40% of the culture volume) was harvested and replaced by an equal volume of fresh growth medium. Cells were grown in 500 mL of modified Chu-13 medium in conical Fernbach flasks upon orbital shaking. The slopes of the straight lines defined the corresponding rates of biomass accumulation, equal to (a) 125 mg dw L⁻¹ d⁻¹ for Botryococcus braunii var. Showa, (b) 80 mg dw L⁻¹ d⁻¹ for Kawaguchi-1, (c) 135 mg dw L⁻¹ d⁻¹ for Yamanaka, (d) 60 mg dw L⁻¹ d⁻¹ for UTEX 2441, (e) 110 mg dw L⁻¹ d⁻¹ for UTEX LB-572, and (f) 195 mg dw L⁻¹ d⁻¹ for Botryococcus sudeticus (UTEX 2629).

FIG. 12. Microscopic observations of a dispersed B. braunii var. Showa micro-colony, showing the grape-seed-like green cells for all B. braunii strains (a-e) and round green cells (f) Botryococcus sudeticus (UTEX 2629). Bars indicate 10 μm.

FIG. 13. In vivo buoyant densities of various live Botryococcus cells, sorted according to increasing buoyant density of the samples. (a) Botryococcus braunii var. Showa, (b) Kawaguchi-1, (c) Yamanaka, (d) UTEX 2441, (e) UTEX LB-572, and (f) Botryococcus sudeticus (UTEX 2629). A 10-80% (w/v) sucrose gradient was employed with a 10% increment among the gradient steps.

FIG. 14. Aqueous buoyant separation of extracellular hydrocarbons from the Botryococcus biomass following sonication of (a) Botryococcus braunii var. Showa, and (b) Botryococcus braunii var. Kawaguchi-1. A 10-80% (w/v) sucrose gradient was employed with a 10% increment among the gradient steps.

FIG. 15 Absorbance spectra of heptane extracts of Botryococcus braunii var. Showa (a and c), and Botryococcus braunii var. Kawaguchi-1 (b and d) micro-colonies. Absorbance of extracts in the blue (380-520 nm) region of the spectrum (a and b) are attributed to extracellular carotenoids from the two strains. Absorbance of extracts in the far UV (190-220 nm) region of the spectrum (c and d) are attributed to extracellular botryococcenes from the two strains, respectively.

### DETAILED DESCRIPTION OF THE INVENTION

### Definitions

The term “terpenoid hydrocarbon” or “isoprenoid hydrocarbon” in the context of this invention refers to terpenoid hydrocarbons formed by combinations of two or more isoprene units. “Terpenoid hydrocarbons” as defined herein include the triterpenoid hydrocarbons botryococcenes and methylated squalenes.

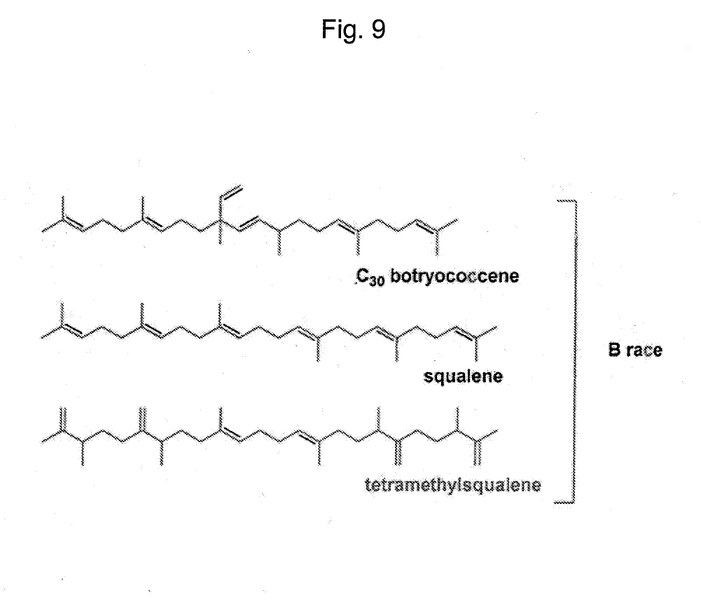

In the context of this invention, “botryococcenes” are triterpenoid C₃₀-C₃₇ hydrocarbons derived from a Botrycocccus terpenoid biosynthetic pathway. An example of a botryococcene structure is provided in FIG. 9.

Also in the context of this invention, “methylated squalenes” are triterpenoid C₃₁-C₃₄ hydrocarbons derived from a Botrycocccus terpenoid biosynthetic pathway. An example of a methylated squalene structure is provided in FIG. 9.

“Botryoxanthin” refers to a carotenoid produced and secreted by Botryococcus.

An algae “micro-colony” refers to an aggregation of green algae cells, e.g., Botryococcus green algae cells, that are held together by a hydrocarbon matrix.

“Mechanical disruption” of algae micro-colonies in the context of this invention refers to use of a physical process, e.g., agitation, sonication, to disrupt and disperse a micro-colony by shear force.

### Algae Micro-Colonies

The invention provides method of extracting terpene hydrocarbons that are produced by the cells and accumulate extracellularly in micro-colonies of green algae. Green algae that are used in the invention typically are members of the genus Botryococcus. However, terpenoid hydrocarbons may be extracted from other micro-colony-forming algae where the hydrocarbons are secreted using methods as described herein.

### Extraction of Hydrocarbons

The invention provides methods of collecting extracellular terpenoid and carotenoid hydrocarbons from green algae micro-colonies. Terpenoids that can be extracted include triterpenoid hydrocarbons such as C₃₀-C₃₇ botryococcenes and C₃₁-C₃₄ methylated squalenes.

Botryococcene hydrocarbons are modified triterpenes that have the chemical formula CₙH₂ₙ₋₁₀. In some embodiments of the invention, extracellular botryococcene hydrocarbons are extracted from Botryococcus sp.

Hydrocarbons are extracted from the algae micro-colonies using a method where the colonies are mechanically dispersed without substantially breaking open the algae cells. As the hydrocarbons are largely present in the extracellular space of the micro-colonies, the majority of the terpenoid and/or carotenoid hydrocarbons produced by the organism can be obtained. In the context of this invention, “without substantially breaking open cells” refers to a dispersion technique where at least 70%, often at least 80% or 90%, of the cells are intact. The integrity of the cells for the purposes of this invention is typically determined using visual inspection with a microscope to look for intact green cells. Resumption of growth by the cells, following collection of the extracellular hydrocarbons, is another method of assessing that the cells, or a substantial portion of them, are intact.

Any method of mechanical dispersion can be employed. For example, in some embodiments, the micro-colonies are shaken or vortexed in an aqueous solution, e.g., water, or in an organic solvent that is being used for extraction. This can be performed, e.g., at agitation of speed of up to about 2700 or about 3200 or about 3500 rpm, or greater, so long as the procedure does not substantially break open the cells. In preferred embodiments, vortexing of the algae in the solution typically takes place in the presence of glass beads, e.g., 1 g of glass bead per 1 g wet cell weight. As appreciated by those of skill in the art, the glass beads can be replaced by many other small, solid, inert substances for this purpose, including, e.g., fine sand, small steel spherical balls, and the like.

Other mechanical dispersal techniques include sonication, or passage through a French Pressure Cell. In this embodiment, sonication is performed at low power (such as, e.g., sonication with a Branson sonifier 3-times for 30 sec in a 50% duty cycle pulse mode, power output 5, with 60 sec cooling intervals in-between) to avoid breaking of the cells. Similarly, passage through a French Pressure Cell is implemented at relatively low pressure (e.g., e.g. 0.5-5 kpsi) to avoid cell rupture.

In some embodiments, a sample comprising green algae micro-colonies is subjected to heat treatment, e.g., of up to about 80°, 90°, 95° or about 100° C. to facilitate separation of the extracellular hydrocarbons from the micro-colony. Heat treatment is typically performed for less than 30 or 20 minutes, e.g., for 10 minutes. Heat treatment can reduce the amount of time the sample is subjected to physical dispersion, e.g., agitation. Thus, in some embodiments, a sample may be vortexed for up to one hour or more. In other embodiments a sample may be heat treated for 10 minutes and then agitated for a time period of less than 30 minutes.

The method employs hexane, heptane, or octane for extraction. Typically the extraction is performed in conjunction with the physical dispersion, e.g., agitation or sonication of the micro-colonies is performed in the solvent; however, in some embodiments, the micro-colonies may be dispersed in an aqueous solution, followed by extraction of the aqueous solution using the solvent. In still other embodiments, the hydrocarbon can be separated from the cellular biomass by flotation in aqueous medium.

### Quantification of Hydrocarbon

The invention also provides a method of quantifying the extracted hydrocarbons using spectrophotometric analysis. Often, the quantification of the extracted hydrocarbons is determined using the following equations:

For botryococcene (Btc) hydrocarbons: [Btc]=[A₁₉₀/ε₁₉₀)×MW_{btc}×V]/m_{dcw}, where the extinction coefficient at 190 nm (ε₁₉₀) is 90±5 mM⁻¹ cm⁻¹. (A=absorbance; MW_{btc}=molecular weight of botryococcene (squalene); V=volume of solvent (heptane, hexane, or octane) used; m_{dcw}=gram dry cell weight of biomass that was extracted).

Carotenoid hydrocarbons such as botryoxanthin are also extracted using the methods described herein and quantified spectrophometrically. In some embodiments, the concentration of botryoxanthin can be calculated using the formula: [Botryoxanthin]=[A₄₅₀/ε₄₅₀)×MW_{btc}×V]/m_{dcw}, where the extinction coefficient at 450 nm (ε₄₅₀) is 165±5 mM⁻¹ cm⁻¹.

### EXAMPLES

The examples described herein are provided by way of illustration only and not by way of limitation. Those of skill in the art will readily recognize a variety of non-critical parameters that could be changed or modified to yield essentially similar results.

### Materials and Methods

### Cell Growth and Culture Conditions

Batch cultures of Botryococcus braunii var. Showa (Nonomura, Jap. J. Phycol. 36:285-291, 1988) were grown in the laboratory in 2 L conical Fernbach flasks. Cells were grown in 500 mL of modified Chu-13 medium (Largeau et al., Phytochem. 19:1043-1051, 1980). Approximately 50 mL of a two-week old B. braunii var. Showa culture was used to inoculate new cultures. Cells were grown at 25° C. under continuous cool-white fluorescent illumination at an intensity of 50 μmol photons m-2 s-1 (PAR) upon orbital shaking at 60 rpm (Lab-Line Orbital Shaker No. 3590). Fernbach flasks were capped with Styrofoam stoppers, allowing for sufficient aeration, i.e., gas exchange between the culture and the outside space.

Growth of B. braunii was measured gravimetrically and expressed in terms of both wet cell weight (wcw, based on packed cell volume measurements) and dry cell weight (dcw) per volume of liquid culture (g L-1). Cell weight analysis was carried out by filtering B. braunii cultures through Millipore Filter (8 μm pore size), followed by washing with distilled water. Excess filter moisture was removed by ventilation. Filters were weighed before and after drying at 80° C. for 24 h in a lab oven (Precision), and dry cell matter was measured gravimetrically. This analysis suggested a dcw/wcw ratio of about (0.125±0.025):1 for B. braunii var. Showa micro-colonies.

### Hydrocarbons Extraction and Separation

Cells were harvested from the liquid media by centrifugation (Beckman Coulter/Model J2-21) at 4,500×g for 10 minutes. Approximately 1 g wet cell weight of B. braunii pellet was mixed with 1 g of glass beads (0.5 mm diameter), and suspended upon addition of 10 mL heptane (HPLC Grade—Fischer Scientific). The cells-in-heptane suspension was vortexed for different periods of time, as indicated, at maximum vortexing speed (Fisher Vortex Genie-2). Following this vortexing, 10 mL of growth medium was added to the mixture, resulting in a prompt aqueous-heptane two-phase partition. The bottom aqueous phase contained cells, whereas the top heptane phase contained the extracted hydrocarbons. The heptane layer was removed and collected for measurement of the absorbance spectra in a UV/Visible spectrophotometer (Shimadzu UV 160U). Prior to spectrophotometric analysis, samples were diluted so that absorbance values at the peak wavelength did not exceed 0.5 absorbance units.

The heptane solution of extractable Showa hydrocarbons was carefully collected and evaporated to dryness under a stream of air for hydrocarbon gravimetric quantitation.

### Chlorophyll Measurements

A known amount of culture pellet was mixed with equal weight of glass beads (0.5 mm diameter) and with a known volume of methanol. The glass bead-methanol-biomass mixture was vortexed until the color of the biomass becomes white, indicating full extraction of intracellular pigments. The crude extract was filtered and the absorbance of the green methanolic phase was measured at 470, 652.4 and 665.2 nm. Total carotenoid, chlorophyll (a+b) content, and the Chl a/Chl b ratio were determined by according to Lichtenthaler & Buschmann In: Wrolstad R E, Ed. Current protocols in food analytical chemistry. New York: John Wiley & Sons Inc. pp. F4.3.1-F4.3.8, 2001).

### Example 1

### Determination of Molecular Extinction Coefficients

The molecular extinction coefficients of squalene and β-carotene were determined under the experimental conditions used in these examples with heptane as the solvent. Heptane was selected as the solvent of choice both because it can remove lipophilic molecules from the growth medium without undue adverse effect on the cells (non-toxic), and also because it does not significantly absorb in the UV and blue regions of the spectrum, where hydrocarbons of interest absorb. This property was not observed with other organic solvents, e.g., methanol, ethanol, isopropyl alcohol, butanol, diethylether, dodecane, and isopropyl-tetradecanoate.

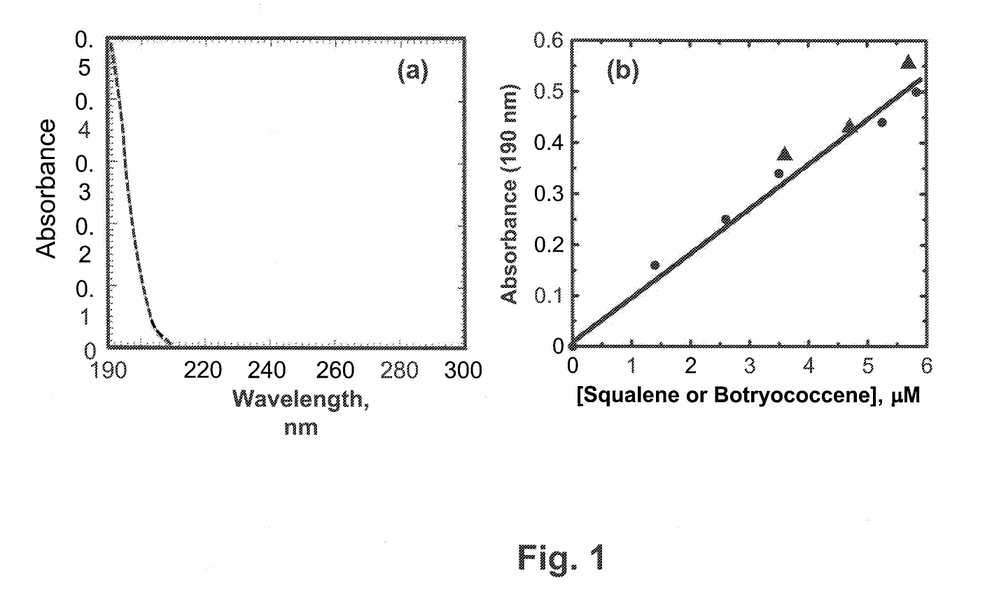

The UV/visible absorbance spectrum of squalene (ACROS Organics, 99% purity) in heptane showed a single absorbance band with a peak at about 190 nm (FIG. 1a). The dependence of this absorbance at 190 nm on the concentration of squalene in heptane was determined in order to obtain the extinction coefficient for this triterpene in this solvent. Absorbance values at 190 nm were measured across a concentration range of 0-10 μM squalene. The slope of the straight line in the measurement of absorbance versus squalene concentration (FIG. 1b, solid circles) defined the molecular extinction coefficient of squalene in heptane at 190 nm to be 90±5 mM⁻¹ cm⁻¹. This squalene extinction coefficient in heptane was somewhat greater than that in acetonitrile, determined by Grieveson et al. (Anal. Biochem. 252:19-23, 1997) to be 59±2 mM⁻¹ cm⁻¹ at 195 nm.

Botryococcene extracts in heptane were also used in quantitative absorbance spectrophotometry. FIG. 1b (open diamonds) shows that absorbance at 190 nm of botryococcene in heptane measured in three different samples and plotted as a function of the botryococcene concentration. The latter was determined gravimetrically upon a subsequent evaporation of the heptane solvent and weighing of the residue in a suitable mg scale. The results suggest that squalene and botryococcene have the same A190 as a function of their concentration in heptane, thus the same extinction coefficient.

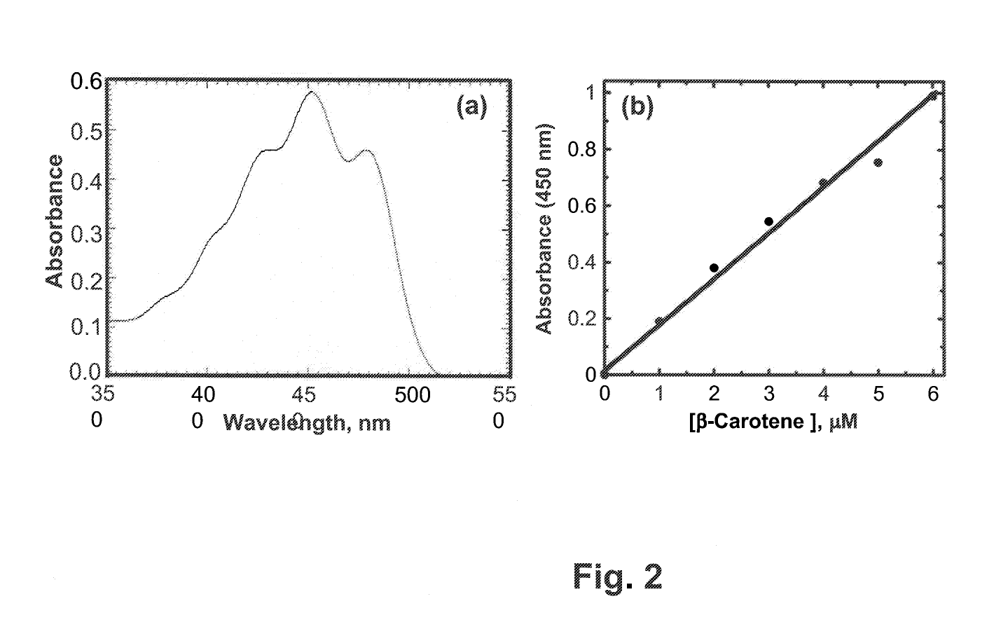

The UV/visible absorbance spectrum of β-carotene (MP Biomedicals) in heptane showed typical features of multiple carotenoid absorbance bands in the blue region of the spectrum (FIG. 2a). The major absorbance band occurred with a peak at 450 nm, with secondary absorbance peaks at 425 and 480 nm. The dependence of the major absorbance at 450 nm on the concentration of β-carotene in heptane was determined in order to obtain the extinction coefficient for this carotenoid in such solvent. Absorbance values at 450 nm were measured across a concentration range of 0-6 μM β-carotene. The slope of the straight line in the measurement of the absorbance versus β-carotene concentration (FIG. 2b) defined the molecular extinction coefficient of β-carotene in heptane at 450 nm to be 165±5 mM-1 cm⁻¹. This β-carotene extinction coefficient in heptane is consistent with results obtained in other solvents. For example, Zhang et al. (J. Biol. Chem. 274:1581-1587, 1999) reported ε(β-carotene at 450 nm) in hexane to be 134 mM⁻¹ cm⁻¹, whereas Eijckelhoff & Dekker (Photosynth. Res. 52:69-73, 1997) found an ε(β-carotene at 450 nm) in methanol to be 140 mM⁻¹ cm⁻¹. Land et al. (J. Chem. Soc. D-Chem. Commun. 6:332, 1970) previously reported on the extinction coefficient of β-carotene in hexane, in the wavelength region of 515 nm, to be 170±40 mM⁻¹ cm⁻¹.

Absorbance spectra of β-carotene in heptane were extended from the blue through the low UV region, down to 190 nm. The A190/A450 ratio was determined to be about 4:1 for this pigment (not shown). Determination of this ratio was important in order to properly partition A190 measurements between botryococcenes and carotenoids in the heptane Showa extracts.

### Example 2

### Micro-Colony Properties of B. Braunii Var. Showa

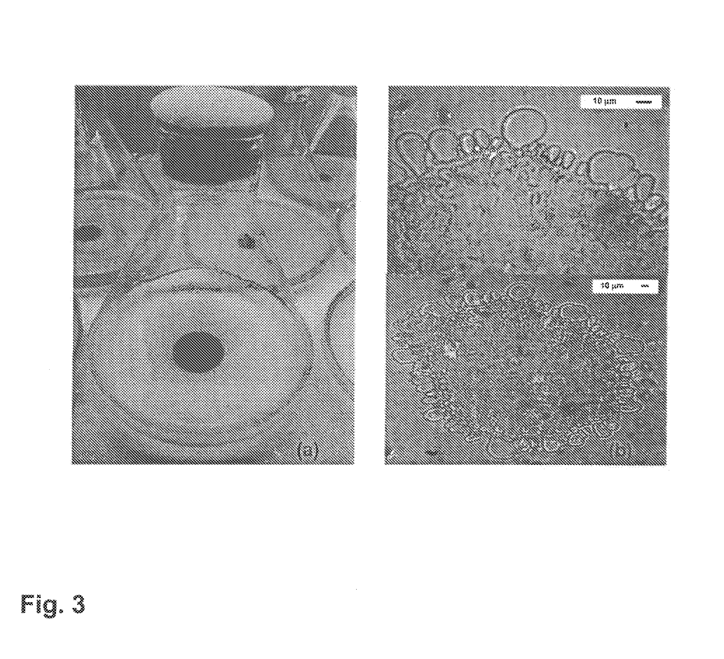

FIG. 3a shows a group of Fernbach flasks with Showa cultures in different phases of growth. Typical in these cultures, and distinct among cultures of other unicellular microalgae, is the tendency of the micro-colonies of Showa to aggregate, or “centrifuge”, toward the center of the growth medium, apparently a result of the orbital shaking of the culture and a consequence of the high hydrocarbon content of these micro-colonies. Showa hydrocarbons can be readily seen in microscopic images of “lightly compressed” micro-colony preparations, in which droplets of botryococcene hydrocarbons are clearly seen effusing from the micro-colony (FIG. 3b).

### Example 3

### Rates of B. Braunii Var. Showa Growth and Productivity

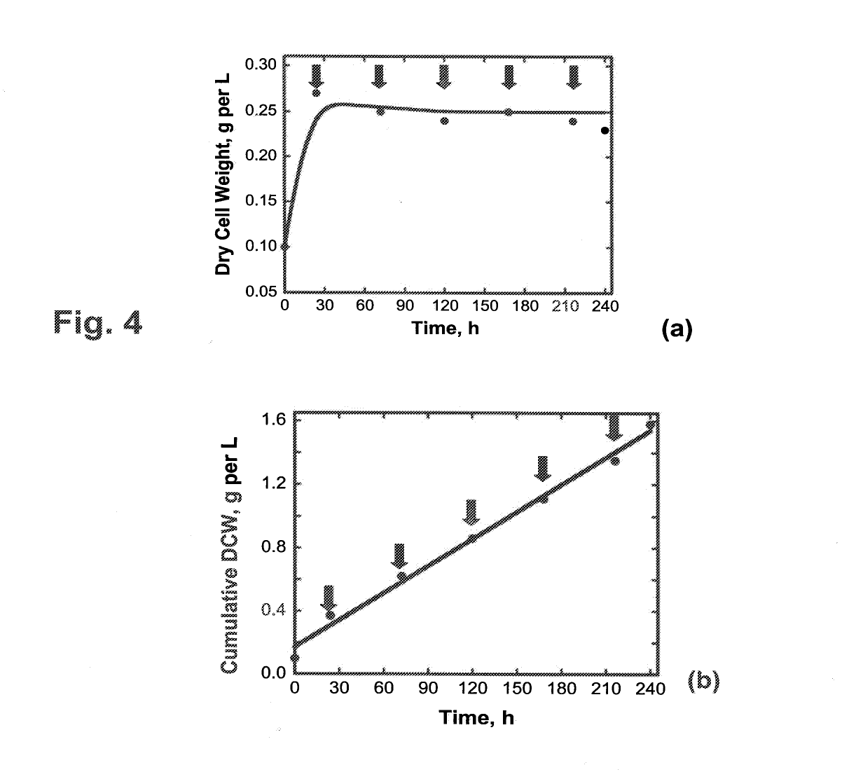

After approximately 10 days of growth in batch culture, Showa cells reached a biomass density of about 200 mg dry cell weight per liter culture. To measure the rate of growth under continuous culturing conditions, 40% volume (200 mL) of the initial culture was removed from the Fernbach flasks and replaced with an identical volume of fresh growth media. This removal-and-replacement was repeated every 48 hours, followed by harvesting by centrifugation and measurement of the biomass. FIG. 4a plots the dry cell weight of the harvested biomass in grams per liter. The results in FIG. 4a suggest a rate of biomass accumulation equivalent to about 250 mg dry cell weight per liter culture per 48 hours, or about 125 mg dcw L⁻¹ d⁻¹. The cumulative dry cell weight from such an experiment is plotted in FIG. 4b. In this continuous growth system, and under the specific growth conditions employed, the slope of the straight line showed an algal biomass increase occurring with a rate of 125 mg dcw L⁻¹ d⁻¹, in agreement with the previous measurement (FIG. 4a). By way of comparison, An et al. (J. Appl. Phycol. 15:185-191, 2003) reported a rate of biomass accumulation of ˜190 mg dcw L⁻¹ d⁻¹ (including about 30 mg botryococcene L⁻¹ d⁻¹) from the culture of Botryococcus braunii UTEX-572, grown in secondarily treated piggery wastewater in a batch reactor. On the other hand, also working with the UTEX-572 strain, grown in secondarily treated sewage in a continuous bioreactor system with a daily dilution rate of 0.57, Sawayama et al. (Appl. Microbiol. Biotechnol. 41:729-731, 1994) measured a biomass production rate of only about 28 mg dcw L⁻¹ d⁻¹. It is evident from these results that B. braunii growth conditions, including bioreactor design and growth media composition, can impact productivity of the cultures.

### Example 4

### Mechanical Dispersion of B. Braunii Var. Showa Micro-Colonies

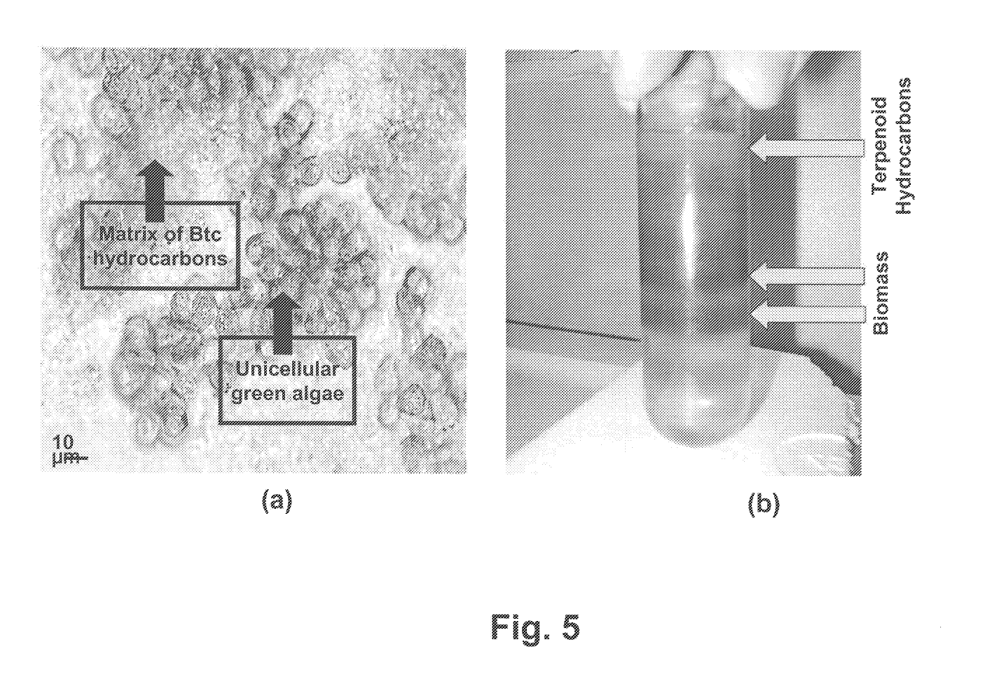

Mechanical dispersion studies of Showa micro-colonies were conducted to test for the behavior of the micro-colonies under such external shearing forces. This was implemented either by sonication, or glass-bead beating of the cultures in growth medium. Microscopic observations of mechanically dispersed Showa micro-colonies (FIG. 5a) revealed extensive disintegration of the normally compact micro-colonies. A substantial extracellular yellowish matrix (FIG. 5a, Btc) was largely separated from the grape-seed-like green cells. Interestingly, Showa cells appeared to retain their intactness, in spite of the mechanical dispersion of the otherwise tightly formed micro-colony. Nile red staining confirmed the lipophilic nature of the colony-surrounding Btc matrix and further revealed intracellular globules of highly lipophilic matter, presumably sites of botryococcene sequestration. The results shown in FIG. 5a demonstrate that the majority of the botryococcenes are extracellularly localized. These results are consistent with findings by Wolf et al. (supra, 1985), who estimated that only about 7% of the botryococcenes are intracellular, with the majority of these hydrocarbons forming the extracellular colonial matrix. Likewise, Largeau et al. (supra, 1980) reported that 95% of the botryococcenes are located in the extracellular pool of hydrocarbons.

A simple centrifugation in sucrose gradient of the mechanically dispersed micro-colonies was performed to determine if mechanical dispersion was sufficient to dislodge the hydrophobic botryococcene-carotene hydrocarbons from the extracellular matrix of the micro-colonies. Centrifugation in sucrose gradient was recently designed to provide a measure of the buoyant density of biomass upon measurement of the “density equilibrium” of the sample (U.S. patent application Ser. No. 12/215,993; Eroglu and Melis, Biotechnol. Bioeng. 102:1406-1415, 2009). The outcome of such a sucrose density centrifugation, conducted with mechanically dispersed Showa micro-colonies, is seen in FIG. 5b. Surprisingly, the results showed a clear-cut separation of the yellowish hydrocarbons (FIG. 5b), which floated on top of the 10% sucrose step, from the of B. braunii green biomass that equilibrated in the vicinity of the 40-50% sucrose step.

### Example 5

### Determination of the Hydrocarbon Productivity in B. Braunii Var. Showa Cultures

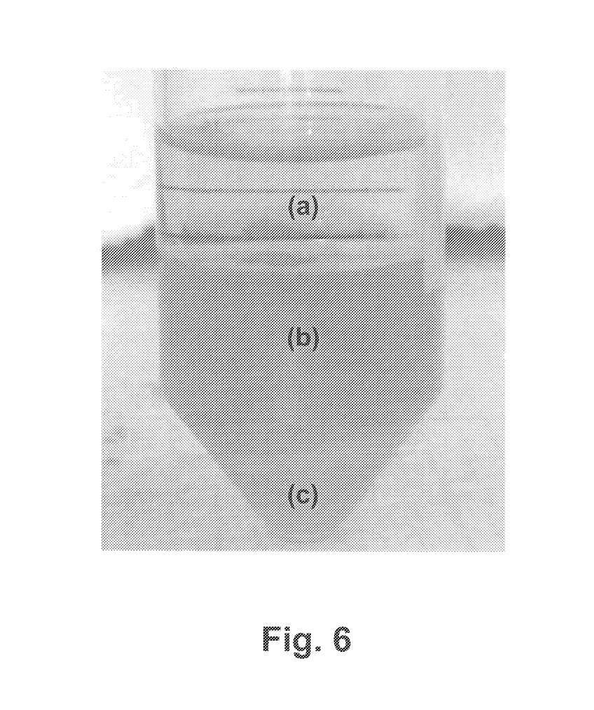

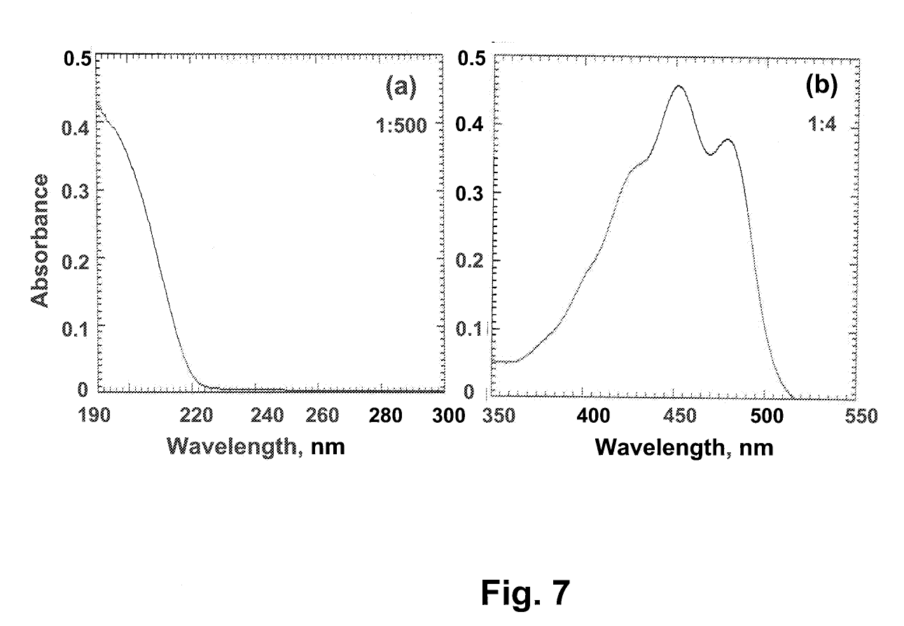

The preceding mechanical dispersion experiment in Example 4 suggested that one should be able to selectively extract botryococcene and related hydrocarbons from the extracellular matrix of the micro-colonies. Vortexing of Showa biomass with glass beads in the presence of heptane resulted in a release of extracellular hydrocarbons from the micro-colony and their subsequent solubilization in the heptane phase. FIG. 6 shows the outcome of such an extraction experiment, in which the top heptane phase (FIG. 6a) contains a clear yellowish solution, whereas the lower water phase contains the green cell biomass (FIG. 6b). The glass beads are also seen in the bottom of the Falcon tube (FIG. 6c). Measurement of the absorbance spectrum of this heptane extract is shown in FIG. 7. Two distinct and separate absorbance bands were discerned, one peaking in the UV-C (λmax=˜190 nm) attributed to botryococcene hydrocarbons, the second in the blue region of the spectrum (380-520 nm), attributed to a carotenoid, apparently associated with the extracellular hydrocarbons in B. braunii. Of interest is the lack of green pigmentation and absence of chlorophyll absorbance bands in this spectrum, consistent with the notion that the heptane-extracted hydrocarbons originated from the extracellular space and not from components of the photosynthetic apparatus from within the cell. The amplitude ratio A190/A450 of the Showa extracts in heptane was measured to be in the range of 110:1; i.e., substantially greater than the 4:1 attributed to the absorbance of a carotenoid. On the basis of these spectrophotometric measurements, and the extinction coefficients provided from the results of FIG. 1b and FIG. 2b, a [Btc]/[Car]=200:1 mol:mol ratio was determined ([Car]:[Btc]=0.5:100 mol:mol).

The chlorophyll content of the cells, and total carotenoid content of the micro-colonies was measured, following the methanol extraction and spectrophotometric quantitation method of Lichtenthaler & Buschmann (supra, 2001). Total chlorophyll (a+b) was found to be 5±1 mg per g dcw (0.5±0.1% w/dcw), and the Chl a/Chl b ratio was 2.2:1 (±0.2). This Chl content of the cells is similar to that reported in the literature. For example, measurements by Singh & Kumar (World J. of Microbiol. and Biotechnol. 8:121-124, 1992) showed a Chl a content for B. braunii cultures under optimum and nitrogen-deficient conditions in batch cultures to be 0.7% and 0.4% of dry cell weight, respectively.

Total carotenoid content of the Showa cultures was 2.5±1 mg per g dcw (0.25±0.1% w/dcw), translating into a Chl/Car ratio around 2:1 (w/w). This carotenoid quantitation includes both extracellular carotenoids, associated with the botryococcene fraction, and thylakoid membrane carotenoids, associated with the photosynthetic apparatus.

Application of the molecular extinction coefficients of botryococcene and β-carotene in heptane (FIGS. 1b and 2b, respectively) provided a direct and convenient way for the quantitative measurement of the amount of these hydrocarbons, extracted from B. braunii micro-colonies by the glass bead method. In the present study, the amounts of botryococcene (Btc) and carotenoid (Car) extracted from Showa cultures were calculated on the basis of the following equations:

[Btc]=[(A190/ε190)×MW_{Btc}×V]/m_{dcw} (1)

[Car]=[(A450/ε450)×MW_{Car}×V]/m_{dcw} (2)

where [Btc] and [Car] are given in μg per g dcw; A=Absorbance; ε=molar extinction coefficient for botryococcene (190 nm) and carotene (450 nm); MW_{Btc}=Molecular weight of squalene (411 g/mol); MW_{Car}=Molecular weight of β-carotene (537 g/mol); V=volume of heptane used for extraction (mL); and m_{dcw}=amount of biomass that was subjected to extraction (gram dry cell weight).

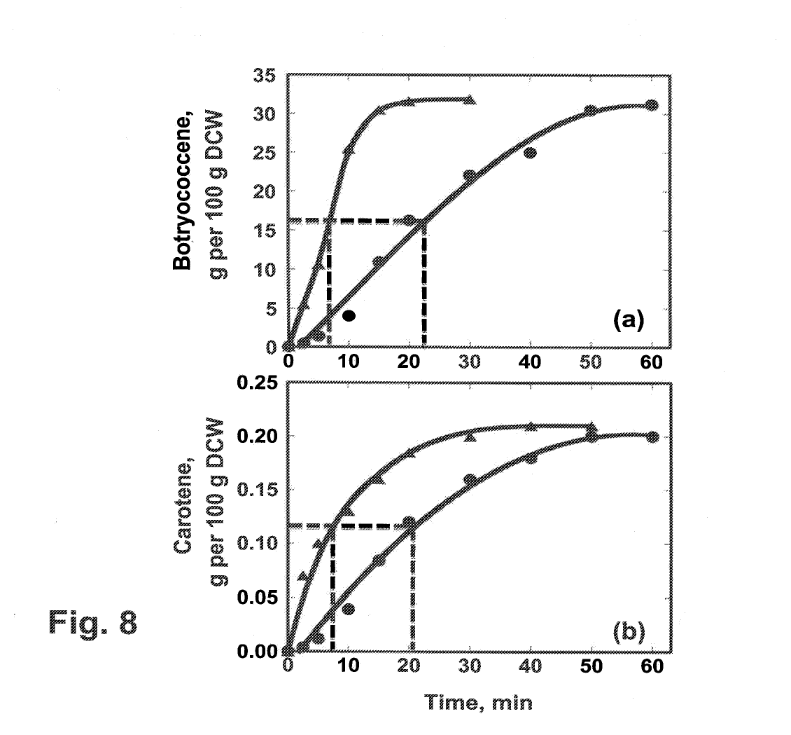

FIG. 8a shows the time-course of the amount of botryococcene extracted in control samples (circles) and samples incubated at 100° C. for 10 min (triangles), as a function of vortexing time in the presence of heptane and glass beads. It is evident from these results that increasing amounts of botryococcene are extracted from the micro-colonies as a function of vortexing time, reaching 0.32 g Btc per g dcw (32% w/dcw). Heating the samples to 100° C. for 10 min prior to vortexing enhanced the efficiency of Btc extraction and shortened the time needed for extraction of these hydrocarbons by the factor of about 3.5. FIG. 8b also shows increasing amounts of carotene extracted from the micro-colonies, as a function of vortexing time, reaching 0.0022 g Car per g dcw (0.22% w/dcw). Heating of the samples to 100° C. for 10 min prior to vortexing enhanced the efficiency of Car extraction and shortened the time needed for extraction of these hydrocarbons by the factor of about 3.3, consistent with the results obtained in the extraction of Btc.

These results, based on the spectrophotometric absorbance analysis are consistent with gravimetric measurements of extracts from the Showa strain (not shown) and also with previously reported results. For example, Wolf et al. (supra, 1985) reported that Showa accumulates 24-29% of its dry biomass in the form botryococcene hydrocarbons. Yamaguchi et al. (Agric. Biol. Chem. 51:493-498, 1987) measured 34 g hydrocarbons per 100 g dcw from the “Berkeley” strain, i.e., Showa. Nonomura (supra, 1988) reported a greater Btc hydrocarbon content in Showa (about 30% w/dcw) than in other strains of B. braunii (1.5 to 20%). Okada et al. (J. Appl. Phycol. 7:555-559, 1995) estimated that the B-race of B. braunii micro-colonies accumulate hydrocarbons in the range of 10-38% of dry cell weight. The presence of a carotenoid that co-extracts with botryococcene hydrocarbons from B. braunii cultures has also been reported. Thomas et al. (Screening for lipid yielding microalgae: Activities for 1983. Final Subcontract Report, Solar Energy Research Institute, USA 1984) reported carotenoid formation ranging between 0.22-0.48% w/dcw in B. braunii UTEX-572. Rao et al. (Bioresour. Technol. 98:560-564, 2007) estimated the content of extractable carotenoid pigments to be about 0.25% w/dcw in B. braunii UTEX-572. Carotenoid accumulation relative to biomass may depend on the “age” of the culture. For example, cells in the stationary phase having a brownish coloration might contain greater relative amounts of this pigment than actively growing cells that usually appear to be green (Largeau et al., supra, 1980).

It was also reported that carotenoids covalently bound to botryococcenes might form the extracellular matrix in some of the Botryococci species (Okada et al., Tetrahedron 53:11307-11316, 1997). The modified extracellular carotenoid was termed “botryoxanthin”, implying stoichiometric parity between botryococcenes and botryoxanthins. However, it is evident from our results that botryococcene hydrocarbons far outnumber any such carotenoids in the extracts of Botryococcus braunii var. Showa.

These examples thus provide experiments that demonstrated that separation of botryococcene hydrocarbons from the Botryococcus micro-colonies can be achieved mechanically, upon vortexing of the micro-colonies with glass beads, either in water followed by buoyant density equilibrium to separate hydrocarbons from biomass, or in the presence of heptane as a solvent, followed by aqueous/organic two-phase separation of the solubilized hydrocarbons (upper heptane phase) from the biomass (lower aqueous phase).

Spectral analysis of the upper heptane phase revealed the presence of two distinct compounds, one absorbing in the UV-C, attributed to botryococcene(s), the other in the blue region of the spectrum, attributed to a carotenoid. Specific extinction coefficients were developed for the absorbance of triterpenes at 190 nm (ε=90±5 mM⁻¹ cm⁻¹) and carotenoids at 450 nm (ε=165±5 mM⁻¹ cm⁻¹) in heptane. This enabled a direct spectrophotometric quantitation of heptane-extractable botryococcenes and carotenoid from B. braunii var. Showa cultures. It was thus estimated that B. braunii var. Showa constitutively accumulates extractable (extracellular) botryococcenes (about 30% of its dry biomass, weight/weight) and a carotenoid (about 0.2% of its dry biomass, weight/weight). It was further demonstrated that heat-treatment of the Botryococcus biomass substantially accelerates the rate and yield of the extraction methods.

### Example 6

### Comparison of Methods for Quantifying Hydrocarbon Productivities in Microalgae Strains

In this example, six different Botryococcus strains (two B-Race, and four A-Race) were compared by morphology, productivity and hydrocarbon accumulation. A variety of methods of to assess hydrocarbon productivity were employed, including density equilibrium, spectrophotometry and gravimetric approaches for multiple independent quantifications of B. braunii biomass and yield of hydrocarbon accumulation. The results showed yields of hydrocarbon accumulation by B-race strains of B. braunii substantially greater than those of A race. Moreover, botryococcene hydrocarbons of the B-race could be readily and quantitatively separated from the biomass. Further, results from the comparative analyses in this work showed that botryococcene triterpenoid hydrocarbon accumulation by B-race microalgae is superior to that of diene and triene accumulation by A-race microalgae, both in terms of yield and specificity of hydrocarbon separation from the biomass.

The materials and methods for this example are as follows:

### Organisms, Growth Conditions, and Biomass Quantitation

Cells of six different Botryococcus species and Chlamydomonas reinhardtii were grown in 500 mL of modified Chu-13 medium (Largeau et al., supra, 1980) in 2 L conical Fernbach flasks. Botryococcus braunii var Showa was obtained from the University of California (UC Berkeley Herbarium Accession No UC147504) (Nonomura, supra, 1988). Botryococcus braunii strains Kawaguchi-1 and Yamanaka were obtained from the University of Tokyo (Okada et al., supra, 1995). Botryococcus braunii UTEX 2441, UTEX LB-572 and B. sudeticus UTEX 2629 were obtained from the culture collection of the Univ. of Texas. Cells were grown at 25° C. under continuous cool-white fluorescent illumination at an incident intensity of 50 μmol photons m⁻² s⁻¹ (PAR) upon orbital shaking of the Fernbach flasks at 60 rpm (Lab-line Orbit Shaker No. 3590). Flasks were capped with Styrofoam stoppers, allowing for sufficient aeration, i.e., gas exchange between the culture and the outside space. Two-week old cultures were used to inoculate new cultures, such that the starting cell concentration of the newly inoculated culture was at about 0.1 g dry weight (dw) per liter. To measure the rate of growth under continuous-fed growth conditions, a fixed fraction of the culture (40% of the total volume) was periodically removed from the Fernbach flasks and replaced by an equal volume of fresh growth medium. Dry cell weight and hydrocarbon content of the harvested biomass, measured in grams per liter of harvested volume, was plotted as a function of time. The frequency of culture harvesting and medium replacement was 24 h for Botryococcus sudeticus (UTEX 2629), 48 h for Botryococcus braunii var. Showa, Botryococcus braunii var. Yamanaka, Botryococcus braunii var. UTEX LB-572, and 72 h for Botryococcus braunii var. Kawaguchi-1 and Botryococcus braunii var. UTEX 2441.

Algal growth and biomass accumulation was measured gravimetrically and expressed in terms of dry weight (dw) per volume of culture (g L⁻¹). Dry cell weight analysis was carried out upon filtering the samples through Millipore Filter (8 μm pore size). The cell weight was measured as recently described (Eroglu and Melis, Bioresource Technology, 101(7):2359-2366, 2010), after drying the filters at 80° C. for 24 h in a lab oven (Precision), and measurement of the dry cell matter (dw). When applied, dispersion of the microcolonies was achieved by sonication of the samples for 4 min with a Branson sonifier, operated at a Power output of 7 and 50% duty cycle (Eroglu and Melis, supra, 2009). Sonication processes were carried out at 4° C.

### Density Equilibrium Measurements

Sucrose density gradient centrifugation of culture aliquots, spanning a sucrose concentration range from 10-80% (w/v), and having a concentration increment step of 10%, were prepared. Sucrose was dissolved in a solution containing 10 mM EDTA and 5 mM HEPES KOH (pH 7.5). Sucrose solutions were set in the gradient, as recently described in work from this lab on the application of the density equilibrium concept for hydrocarbon quantifications (Eroglu and Melis, supra, 2009). Samples containing microcolonies, single cells, or subcellular particles of interest, were carefully layered on top of the preformed gradient, followed by centrifugation of the polyallomer tubes in a JS-13.1 swing bucket Beckman rotor, at an acceleration of 20,000 g for 30 min at 4° C. The density equilibrium position of the samples was noted at the end of this centrifugation. Sonication of samples, when appropriate, was applied for 4 min with a Branson sonifier, operated at a power output of 7 and 50% duty cycle.

### Spectrophotometric Quantification of Hydrocarbons

Botryococcus cells were harvested from the liquid media by filtration. Approximately 1 g cake of Botryococcus wet weight (ww) was incubated at 100° C. for 10 min. Following the heat treatment, the cell cake was mixed with 1 g of glass beads (0.5 mm diameter), and resuspended in 10 mL of heptane (HPLC Grade—Fischer scientific). The cells-in-heptane suspension was vortexed for 15 min at maximum speed (Fisher Vortex Genie-2). Vortexing of Botryococcus biomass with glass beads in the presence of heptane resulted in a release of extracellular hydrocarbons from the micro-colony and their subsequent solubilization in the heptane phase. Following aqueous/organic two-phase partition (Eroglu and Melis, supra, 2010), the upper heptane phase was collected for measurement of the absorbance spectra in a UV/Visible spectrophotometer (Shimadzu UV1800). Extractable triterpenoid (botryococcene) hydrocarbons were determined from the absorbance in the UV-C region (λmax=˜190 nm), whereas associated carotenoids were determined from the absorbance of the heptane solution in the blue region of the spectrum (λmax=˜450 nm). Total amounts of botryococcene (Btc) and carotenoid (Car), extracted from the various Botryococcus cultures were calculated on the basis of molar extinction coefficients ε for botryococcene (ε190 nm=90±5 mM⁻¹ cm⁻¹) and carotenoids (ε450 nm=165±5 mM⁻¹ cm⁻¹) (Example 5).

### Spectrophotometric Quantitation of Chlorophyll (Chl) and Carotenoid (Car) Content

A known amount of culture pellet was mixed with a known volume of methanol. The methanolbiomass mixture was vortexed at high speed until the color of the biomass became white, indicating full extraction of intracellular pigments. The crude extract was filtered and the absorbance of the green methanolic phase was measured at 470, 652.4 and 665.2 nm. Total carotenoid, chlorophyll (a+b) content, Chl a/Chl b and the Car/Chl ratios were determined according to Lichtenthaler and Buschmann (2001).

### Gravimetric Quantitation of Lipophilic Extracts

The total methanol extract of cells was carefully collected and evaporated to dryness under a stream of air for gravimetric quantitation. Such extract contains all lipophilic cellular compounds, including diglycerides (DG), Chl, Car, and potentially accumulating hydrocarbons. The amount of accumulating hydrocarbons was estimated upon subtracting the diglycerides (DG), Chl, and Car content from the overall lipophilic cell extracts. This was accomplished upon consideration of a known (and constant among microalgae) DG/Chl ratio, derived for the model microalga Chlamydomonas reinhardtii. The latter does not accumulate terpenoid or alkadiene hydrocarbons. Hence, the vast majority of acyl-glycerols in C. reinhardtii are DGs.

### Statistical Analyses

Statistical analysis of the results is based on three independent measurements. Results are expressed as a mean±standard deviation of these 3 independent measurements.

### Results

### Cell Growth

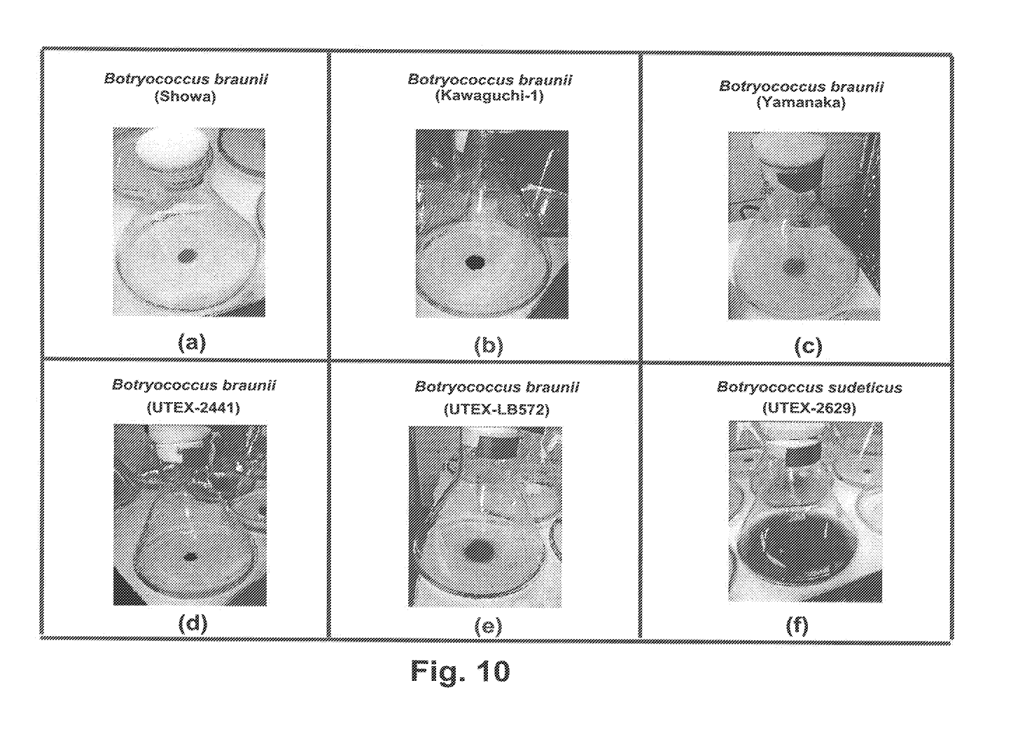

Orbital shaking of Botryococcus cultures in conical Fernbach flasks causes hydrocarbon-laden microcolonies to “centrifuge” to the center of the flask, leaving a clear growth medium in its surroundings. FIG. 10 shows a photograph of a group of Fernbach flasks, taken while on an orbital shaker with various Botryococcus cultures. It is seen that cultures of Botryococcus braunii var. Showa (FIG. 10a), Kawaguchi-1 (FIG. 10b), Yamanaka (FIG. 10c), UTEX 2441 (FIG. 10d), and UTEX LB-572 (FIG. 10e) all “centrifuge” to the center of the 500 mL growth medium. Conversely, FIG. 10f shows a culture of Botryococcus sudeticus (UTEX 2629), in which the cell suspension is uniform throughout the liquid medium during orbital shaking.

The tendency of the micro-colonies to segregate toward the center of the growth medium upon orbital shaking (FIG. 10) is apparently a result of the centrifugal forces applied to the culture and a consequence of the hydrocarbon content of the micro-colonies. This contention is supported by observations of other microalgae that do not accumulate hydrocarbons. For example, Chlamydomonas reinhardtii with a thick cell wall, and Dunaliella salina with no cell wall, having substantially different “Density Equilibrium” properties (Eroglu and Melis, supra, 2009), when cultivated under similar orbital conditions, both showed a cell suspension uniformly dispersed throughout the liquid medium (not shown). Further, B. braunii var. Showa microcolonies and cells, from which botryococcene hydrocarbons were removed, became uniformly dispersed in the growth medium upon orbital shaking. It may be inferred that B. sudeticus, with cells uniformly dispersed in the growth medium (FIG. 10f) does not accumulate hydrocarbons to the same extend as the case is with the B. braunii strains.

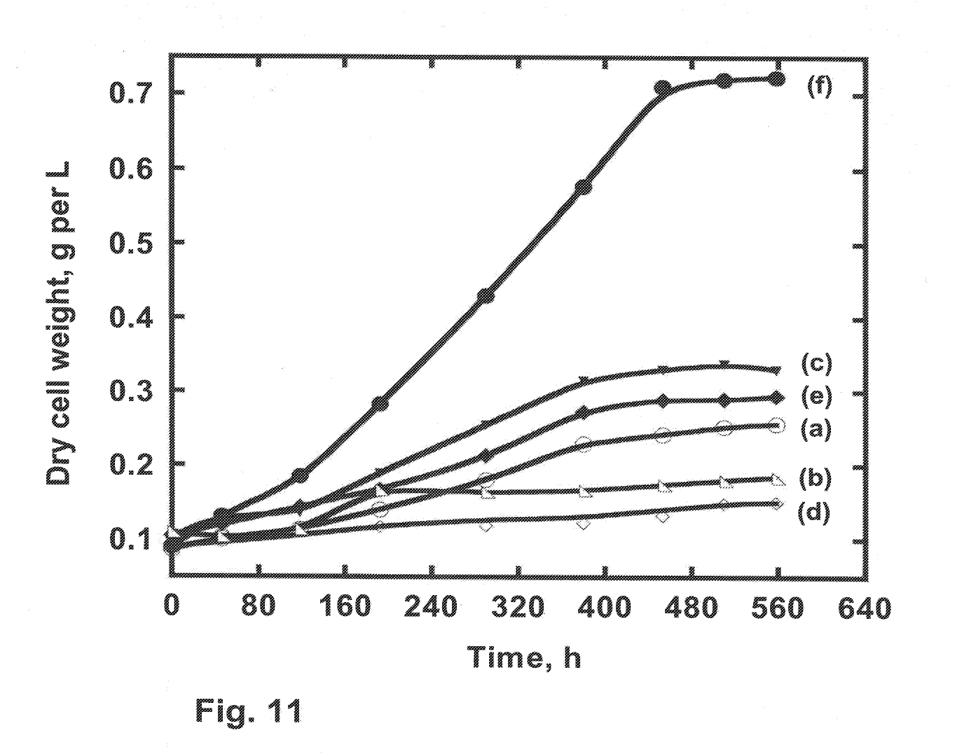

Growth rates of the different Botryococci strains were obtained upon cultivation under identical conditions in continuous fed cultures. Biomass accumulation was measured upon periodic removal of a fixed fraction of the culture (40% of the culture volume) and replacement by an equal volume of fresh growth medium. Under these conditions, cultures remained in an active growth phase. Productivity estimates were based on the volume of growth medium that was used-and-replaced in this continuous-fed process, rather than on the steady state total volume of the culture. The rationale for choosing this basis for the productivity of the culture is that, in a commercial hydrocarbons-production continuous-fed process, costs associated with the replacement volume would figure prominently, not so much those of the steady-state volume of the culture. The cumulative dry cell weight of the biomass from each Botryococcus strain was measured in grams per liter and plotted in FIG. 11. Rates of biomass accumulation, obtained from the slopes of the straight lines, revealed that B. sudeticus accumulated dw with a rate of 195 mg dw L⁻¹ d⁻¹, FIG. 11f), B. braunii var. Yamanaka accumulated dw with a rate of 135 mg dw L⁻¹ d⁻¹, FIG. 11c), followed by Showa (125 mg dw L⁻¹ d⁻¹, FIG. 11a), UTEX LB-572 (110 mg dw L⁻¹ d⁻¹, FIG. 11e), Kawaguchi-1 (80 mg dw L⁻¹ d⁻¹, FIG. 11b) and UTEX 2441 (60 mg dw d⁴, FIG. 11d).

### Micro-Structural Organization of Botryococcus Strains

Botryococcus braunii B-race typically have amorphous three-dimensional micro-colony structures, characterized by a botryoid appearance of the micro-colony, where individual grape seed-like, or pyriform-shaped cells are held together by a surrounding hydrocarbon matrix (Metzger and Largeau, supra, 2005; Eroglu and Melis, supra, 2010). These micro-colonies can grow in size to reach up to 1 mm in diameter (Bachofen, Experentia 38:47-49, 1982). The bulk of the B. braunii hydrocarbons are stored within the outer cell walls and in the extracellular spaces of the micro-colony structure (Largeau et al., supra, 1980). Wolf and co-workers (Wolf et al., supra, 1985) calculated that only approximately 7% of the botryococcenes are intracellular with the majority of the microcolony hydrocarbons forming an extracellular matrix. Likewise, Largeau et al. (supra, 1980) reported that 95% of the botryococcenes are located in the extracellular pool of hydrocarbons.

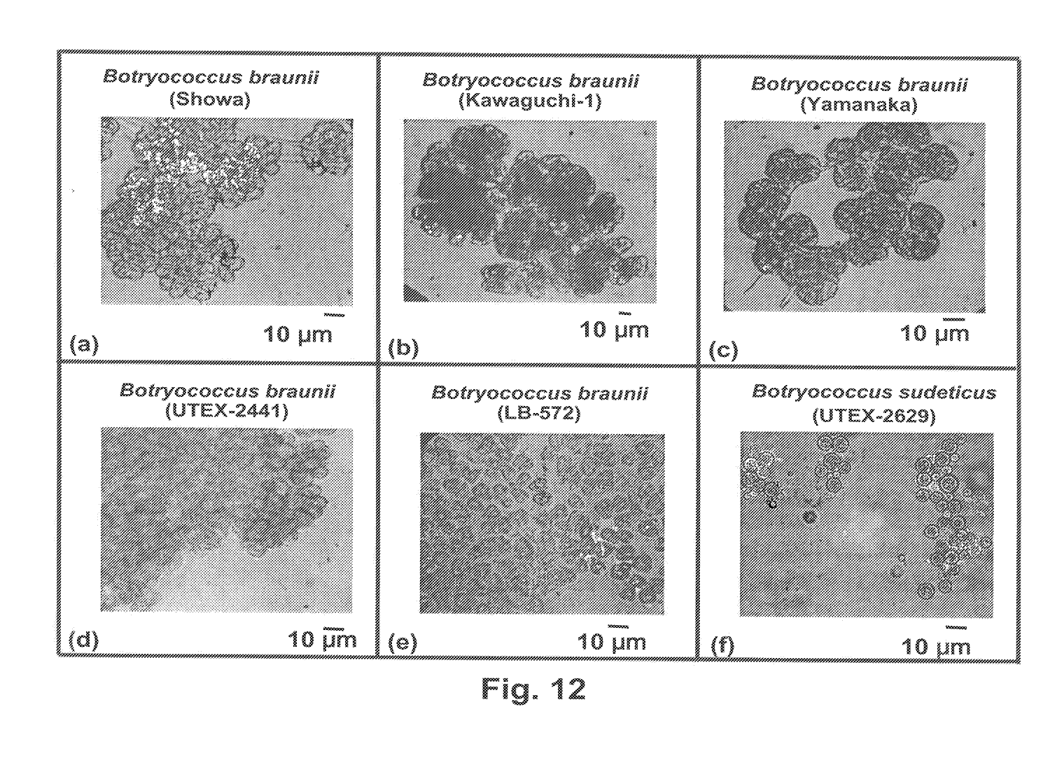

However, there is morphological heterogeneity between the different strains of Botryococcus-type microalgae. Microscopic examination of the strains discussed in this work (FIG. 12 a-f) showed variations both in the size and shape of the cells, which can be more or less embedded in the matrix, and by the presence or absence of refracting threads, forming pili-like structures and clearly linking clusters of cells, thus leading to the formation of large colonies. These characteristics are clearly seen in the B-race of B. braunii, e.g. Showa (FIG. 12a), and Kawaguchi (FIG. 12b), they are also discernible in the A-race of B. braunii, e.g. Yamanaka (FIG. 12c), but are less well developed in UTEX 2441 (FIG. 12d), and LB-572 (FIG. 12e). Botryococcus sudeticus (UTEX 2629) has a distinctly different cell shape from all of the preceding strains, consisting of perfectly spherical single cells without any apparent connectivity among them (FIG. 12f). It is noted that on the basis of rRNA sequencing, Senousy et al. (J. Phycol. 40:412-423, 2004) classified Botryococcus sudeticus in Chlorophyceae, suggesting that it belongs to a genus altogether different than the Botryococci. Microscopic visualization of strains in FIG. 12 will help the field in the proper identification of their Botryococcus samples, and will alleviate the often-erroneous treatment of invading green microalgae in scale-up cultures as part of the Botryococcus biomass.

### Density Equilibrium Properties of Botryococcus Colonies

Wet biomass cake (ww) and dry biomass weight (dw) analysis was carried out by filtering microalgal cultures through Millipore Filter (8 μm pore size), followed by rinsing with distilled water and drying of the filters in a lab oven. This quantitative analysis provided a measure of the dw/ww ratios for each of the Botryococcus strains examined. Chlamydomonas reinhardtii strain CC503 was employed in this experimentation as a control. With the exception of Kawaguchi and UTEX LB572, all other strains had dw/ww ratios of 0.24 (±0.06):1 w/w (Table 1). These microalgal dw/ww ratios are greater from those measured with plant cells (Park and Kim, Biotechnol. Tech. 7:627-630, 1993), reflecting the high-density biomass and the lack a sizable water-filled vacuoles in microalgae. Table 1 also shows that UTEX LB-572 appeared to have a rather low dw/ww ratio 0.08 (±0.02):1 w/w, whereas Kawaguchi-1 appeared to have a much higher dw/ww 0.38 (±0.03):1 w/w ratio.

TABLE 1
Dry weight (dw) to wet weight (ww) ratios and Density Equilibrium
values for various Botryococcus species and Chlamydomonas reinhardtii
strain CC503. Density equilibrium values have been determined upon
sucrose gradient centrifugation of live cultures.

| | | Density Equilibrium, |
|---|---|---|
| Strains | dw/ww (g g⁻¹) | ρ_{S}, g mL⁻¹ |
| B. braunii var. Showa | 0.18 ± 0.04 | 1.03 |
| B. braunii (Kawaguchi-1) | 0.38 ± 0.03 | 1.08 |
| B. braunii (Yamanaka) | 0.20 ± 0.02 | 1.16 |
| B. braunii (UTEX 2441) | 0.30 ± 0.02 | 1.20 |
| B. braunii (UTEX LB572) | 0.08 ± 0.02 | 1.26 |
| B. sudeticus (UTEX 2629) | 0.22 ± 0.03 | 1.34 |
| Chlamydomonas reinhardtii | 0.25 ± 0.04 | 1.35 |

The average dw/ww ratio of 0.24 (±0.06):1 w/w is at variance with some previously reported measurements. For example, the dry to wet weight ratio in Chlamydomonas reinhardtii and similar green microalgae was reported to be 0.1:1 w/w (Ward, Phytochemistry 9:259-266, 1970). This difference is attributed to the different approaches employed in the wet weight determination of the cells. Filtration and the “wet cell cake” approach would tend to remove more water from the microalgae than centrifugation and wet pellet measurement. This is especially so for the oil containing microalgae, which are naturally difficult to precipitate in any type of centrifugation, resulting in a retention of significant amounts of water by the pellet.

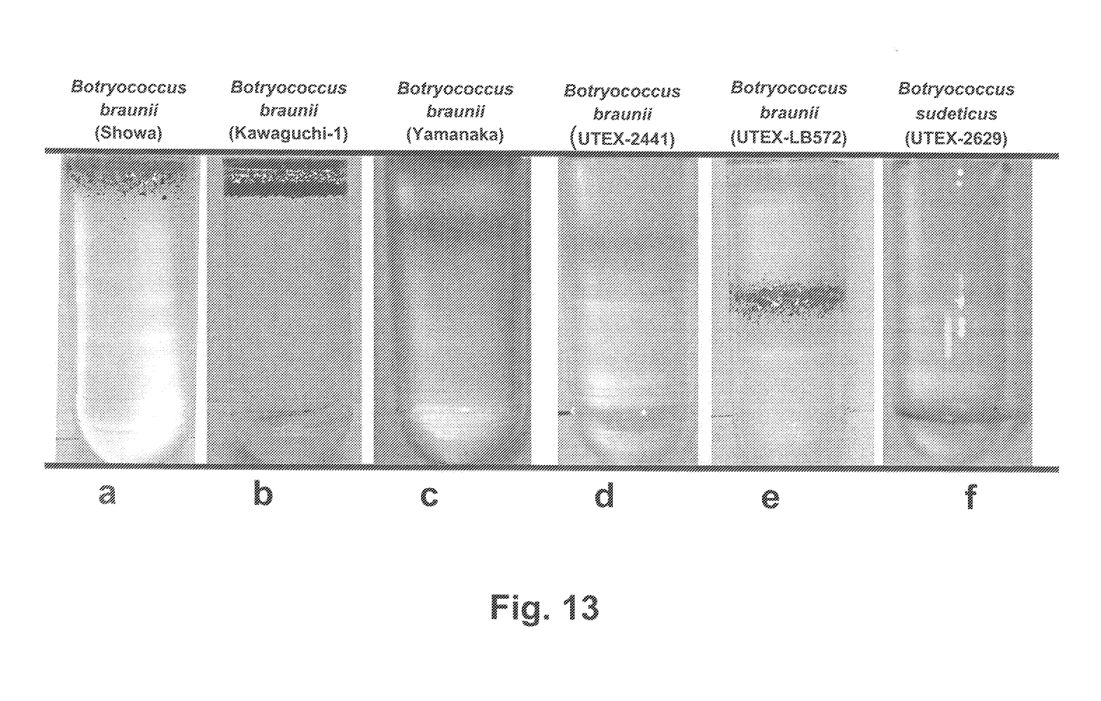

A direct density equilibrium measurement was recently reported, for the rapid in situ estimation of total lipid content in microalgae (Eroglu and Melis, supra, 2009). The method is based on the measurement of the density (ρ) of live cells, or micro-colonies, from which the absolute lipid content of the cells can be calculated. This method was applied with each of the 6 Botryococcus strains examined. FIG. 13 shows the density equilibrium properties of the different strains following centrifugation in a 10-80% sucrose gradient. Two of the B-race strains (Showa and Kawaguchi-1) were the most buoyant among the Botryococci examined. Showa microcolonies floated on top of the 10% sucrose density, i.e. they displayed a density ρ<1.039 g/mL (FIG. 13a). This is consistent with earlier measurements (Eroglu and Melis, supra, 2009), in which Showa micro-colony density was measured more precisely to be ρ=1.031 g/mL. Micro-colonies of Kawaguchi-1 floated at about the 10% sucrose gradient step, i.e., they had an overall density ρ=1.039 g/mL (FIG. 13b). Two different A-race strains (Yamanaka and UTEX 2441) displayed higher ρ values, as their density equilibrium position in sucrose gradient was found to be in the top and bottom boundaries of the 30% sucrose step, respectively (FIG. 13c and FIG. 13d). The calculated absolute density for Yamanaka was approximately ρ=1.10 g/mL (FIG. 13c) and for UTEX 2441 it was ρ=1.14 g/mL (FIG. 13d). Similarly, strain LB-572 (A-race) cells displayed a density equivalent to ρ=1.23 g/mL, as they equilibrated at about the 50% sucrose gradient step (FIG. 13e). On the other hand, Botryococcus sudeticus (UTEX 2629) proved to have the highest density of the samples examined, as it equilibrated at the 70-75% sucrose gradient step, corresponding to a ρ=1.350-1.382 g/mL (FIG. 13f). The density equilibrium values measured for each of the six Botryococcus strains are also summarized in Table 1. This analysis revealed that Botryococcus braunii var. Showa microcolonies have the lowest density equilibrium from the six strains examined. This is consistent with findings by Nonomura (supra, 1988) who provided evidence that Showa differs from other members of the Chlorococcales in terms of the production of high concentrations of liquid hydrocarbons, i.e., C30-C34 botryococcenes (Sato et al., supra, 2003; Okada et al., Arch. Biochem. Biophys. 422:110-118, 2004; Metzger and Largeau, supra, 2005). This property apparently confers to Showa the relatively low-density equilibrium and high buoyancy.

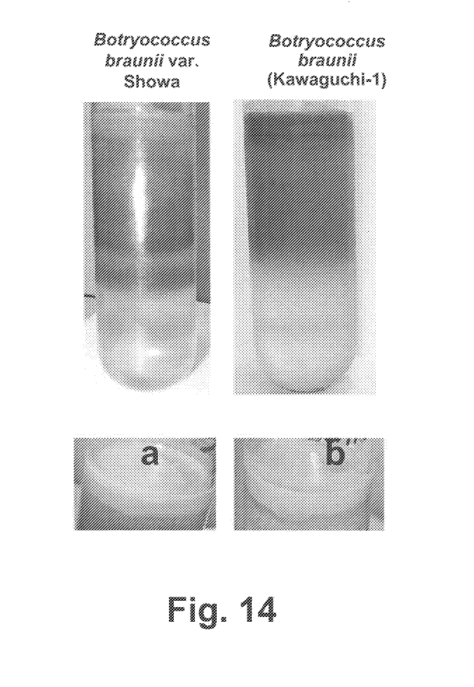

In order to independently measure the contribution of hydrocarbons to the buoyant density of Showa and Kawaguchi-1 strains, a sonication and flotation (Example 4) was employed. In this approach, micro-colonies are mechanically disrupted by sonication or vortexing with glass beads, followed by sucrose density gradient centrifugation. Mechanical dispersion of the micro-colonies dislodges the hydrocarbons form the exterior of the cells, causing the former to float on top of the sucrose gradient. FIG. 14 shows the Density Equilibrium profile of sonicated Showa (FIG. 14a) and Kawaguchi (FIG. 14b) micro-colonies. A yellowish-orange colored hydrocarbons fraction is clearly seen floating on top of the 10% sucrose density step, whereas the B. braunii green biomass equilibrated in the vicinity of the 30-50% sucrose density step, suggesting a cell density of about 1.28 g/mL. Thus, selective removal of the extracellular hydrocarbons from the micro-colonies of the Showa and Kawaguchi strains afforded the cells a much greater density equilibrium property compared to that of the untreated micro-colonies (FIGS. 13a and 13b). The yellow floater band derived from these B. braunii B-race strains, i.e., Showa (FIG. 14a) and Kawaguchi-1 (FIG. 14b) consisted of a mixture of botryococcene and carotenoid, having an altogether density lower than that of water (ρ<1 g/mL). The floating botryococcene fraction of Showa appeared to be more yellow compared to the corresponding orange fraction of Kawaguchi-1 (FIG. 14), probably due to the higher carotenoid content in the latter (see below). The assignments above were further supported by the observation that the amount of the two density equilibrium components (yellow hydrocarbons and green biomass) from these Botryococcus micro-colonies, depended on the amount of sample employed, as well as the duration and power of the mechanical dispersion, suggesting a cause-and-effect relationship between sample size, extent of micro-colony dispersion and the amount of yellow and green product. The results affirm that aqueous density equilibrium can be successfully employed to separate extractable hydrocarbons from the Botryococcus micro-colonies.

Table 2 provides estimates of the amount of hydrocarbon accumulation in Showa and Kawaguchi, based on the “conservation of mass” principle at constant volume and the application of a system of two equations that relate the density equilibrium values of intact microcolonies, floating hydrocarbons and biomass, devoid of the extractable hydrocarbons. This was achieved upon application of the following system of two equations, which relate buoyant densities and relative amounts of biomass and hydrocarbon content in micro-colony samples (Eroglu and Melis, supra, 2009):

ρS=(x·ρP)+(y·ρB) (3)

x+y=1 (4)

Equations (3) and (4) above require experimental measurement of variables such as: ρS; the overall density of the sample, equal to 1.03 g/mL for Showa and 1.08 g/mL for Kawaguchi (Table 1); ρP, the density of the pure hydrocarbon product, equal to 0.86 g/mL for both strains (Eroglu and Melis, supra, 2009); ρB the density of the respective biomass, devoid of the extractable hydrocarbons, equal to 1.28 g/mL for both strains (Table 2); x, is the % fractional weight of the extractable hydrocarbons in the sample; and y, is the % fractional weight of the biomass, devoid of extractable hydrocarbons.

Solution of the above system of equations yielded a 30% and 23% (w/w) botryococcene hydrocarbons content in Showa and Kawaguchi, respectively (Table 2).

TABLE 2
Spectrophotometric determination of extracellular hydrocarbons
from microcolonies of Botryococcus braunii var. Showa
and var. Kawaguchi-1 (B-race).

| | Density Equilibrium | | | |
|---|---|---|---|---|
| | Method | | | |
| | Density of | | Spectrophotometric | |
| | biomass, devoid | | Method | |
| | of Btc, | Btc, | Btc, | Carotenoid |
| Strain | ρ_{B}, (g mL⁻¹) | (% of dw) | (% of dw) | (% of dw) |
| B. braunii var. | 1.28 | 30 | 33 | 0.19 |
| Showa | | | | |
| B. braunii var. | 1.28 | 23 | 21 | 0.49 |
| Kawaguchi-1 | | | | |

A similar differential extraction of hydrocarbons, upon mechanical dispersion of the micro-colonies, and separation from the respective cellular biomass via sucrose density centrifugation could not be achieved with A-race strains. A variety of glass bead and/or sonication regimens were applied but met with mixed results. In this effort, release of hydrocarbons, presumably C25 to C31 odd-numbered n-alkadienes and alkatrienes, occurred in tandem with the release of chlorophyll and other photosynthetic pigments from the cells. A sucrose density centrifugation of such mechanically treated samples resulted in the flotation of hydrocarbons mixed with chlorophyll (not shown). These results suggested that A-race cells, unlike their B-race counterparts, break easily upon mechanical dispersion of the micro-colonies, releasing photosynthetic pigments, which are then mixed with the diene hydrocarbons in the medium.

### Spectrophotometric Determination of Hydrocarbon Content in B-Race Botryococcus Strains

An extraction method of the invention comprising vortexing wet-cake of Showa microcolonies with glass beads in the presence of heptane results in the quantitative release of extracellular hydrocarbons from the micro-colonies, and their subsequent solubilization in the heptane phase, without cell disruption and release of green (Chl) pigments as described herein. This heptane-based differential hydrocarbons extraction approach was successfully applied to both Showa and Kawaguchi strains in this example.

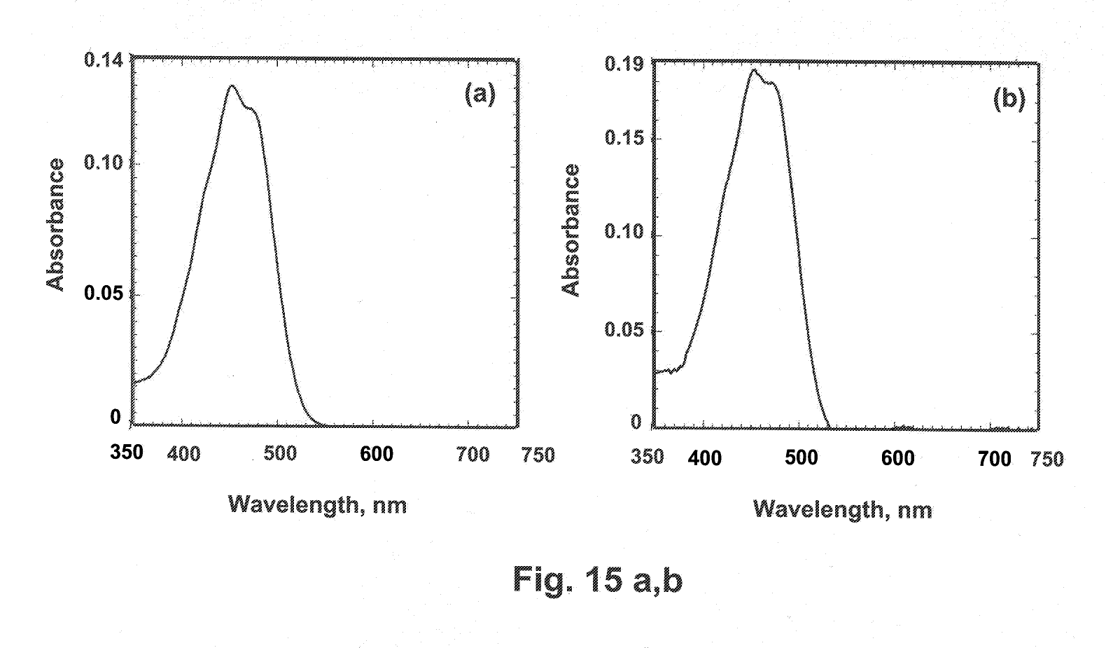

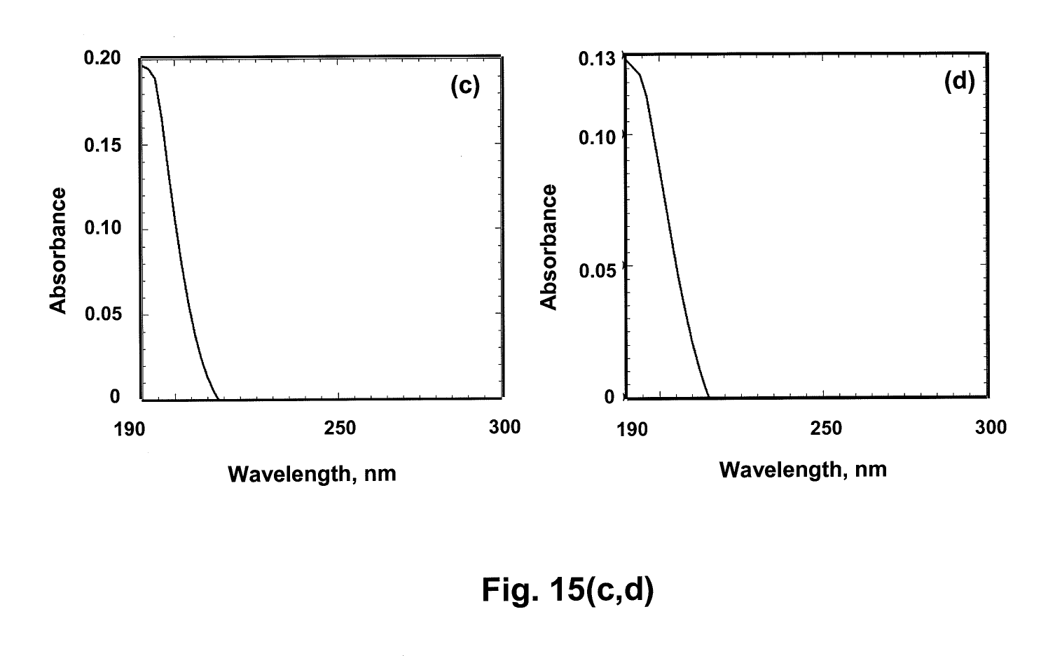

Absorbance spectra of such heptane extracts, measured in the visible region of the spectrum (380-520 nm) showed the presence of a carotenoid with fairly similar absorbance characteristics between the two strains (FIGS. 15a and 15b). The heptane extract also showed a distinct absorbance band in the far UV-C (λmax=˜190 nm) attributed to triterpenoid botryococcenes. The absorbance characteristics of the two UV-C spectra were also fairly similar between the Showa and Kawaguchi (FIGS. 15c and 15d), suggesting presence of the same kind of botryococcenes in the two B-race strains. It was microscopically determined that pyriform shaped B-race strains of Showa and Kawaguchi remained intact, in spite of the mechanical dispersion of the otherwise tightly formed micro-colony and the selective removal of extracellular botryococcene and carotenoids. This specific and quantitative removal and recovery of extracellular hydrocarbons from the B-race strains might serve as basis for the commercial exploitation of B. braunii in the generation and recovery of renewable hydrocarbons.

Only the B-race Showa and Kawaguchi strains were successfully subjected to a selective separation of hydrocarbons from the respective micro-colonies, leaving cells intact in the medium. Attempts at heptane, or other solvent extraction of hydrocarbons from A-race microcolonies were accompanied by the concomitant release of chlorophyll, evidenced by the green coloration in the heptane extract. These results are also consistent with the notion that A-race strains, such as those investigated in this work, are more easily subject to cell rupture and pigment release, compared to their B-race counterparts.

Application of suitable molecular extinction coefficients of the invention permitted quantitative measurement of extracted botryococcenes [Btc] and carotenoids [Car] from the B-race strains. This was achieved upon application of the following equations, which are also provided above in Example 5:

[Btc]=[(A₁₉₀/ε₁₉₀)×MW_{Btc}×V]/m_{dw} (5)

[Car]=[(A₄₅₀/ε₄₅₀)×MW_{Car}×V]/m_{dw} (6)

where, A: Absorbance, ε: molar extinction coefficient for botryococcene (at 190 nm) and carotenoid (at 450 nm) in mM⁻¹ cm⁻¹, MW_{Btc} and MW_{Car}=Assumed molecular weight of botryococcene (410 g/mol) and carotenoid (536 g/mol), respectively, V=volume of heptane used for extraction (mL), m_{dw}=amount of biomass that was subjected to extraction (gram dry cell weight). Solution of Eq. (5) and (6) yields [Btc] and [Car] concentrations in μg per gram dry cell weight. It should be noted that extractable carotenoids from the Botryococcus strains are probably echinenone, botryoxanthin, braunixanthin, or a mixture thereof (Okada et al., supra, 1997; Okada et al., Phytochemistry 47(6):1111-1115, 1998; Tonegawa et al., Fisheries Science 64(2):305-308, 1998). However, molecular extinction coefficients are about the same for most carotenoids and their variants (reviewed by Eroglu and Melis, supra, 2010), justifying the use of a generic extinction coefficient for the Botryococcus carotenoids extracted in the course in this work.

Table 2 (spectrophotometric approach) summarizes the amount of botryococcene that could be extracted from the B-race of Botryococcus species without a concomitant cell lysis. On the basis of these results, it appeared that Showa had a higher content of Btc (33% Btc per dw), whereas Kawaguchi-1 had 21% Btc per dw. Conversely, carotenoid content of the Showa extract was 0.19% of dw, whereas that of Kawaguchi-1 was 0.49% of dw. The substantially greater carotenoid content of Kawaguchi-1 relative to Showa caused the more orangey coloration of these microcolonies (FIG. 13b) and of the extractable hydrocarbons fraction (FIG. 14b). Quantitative results from the spectrophotometric measurements (Table 2, right columns) are consistent with those obtained through the density-equilibrium approach (also Table 2, left columns).

### Gravimetric Determination of Hydrocarbon Content in Microalgae

Analysis of chlorophyll and carotenoid content on per dw basis for the strains examined is given in Table 3. Chlorophyll content was highest for C. reinhardtii (2.05% of dw) and B. sudeticus (1.6% of dw), whereas it was 0.55±0.1% of dw for the B. braunii strains. Thus, B. braunii strains have a lower Chl/dw ratio compared to the unicellular microalgae C. reinhardtii and B. sudenticus. The lower Chl/dw ratio of the former might be a consequence of the unique microcolonial structure and/or due to the accumulation of hydrocarbons in these microalgae.

Regardless of differences in the Chl/dw ratio, all strains examined in this work had similar Chl a/Chl b ratios with an average of 2.3 (±0.5):1 mol:mol (Table 3), suggesting similar organization of their photochemical apparatus (Mitra and Melis, Optics Express 16(26):21807-21820, 2008). Total carotenoid per dw also varied among the strains in a way that was qualitatively similar to that of Chl (Table 3). However, Car/Chl ratios were highest among the hydrocarbon-accumulating B. braunii strains and lowest for the non-accumulating strains, including C. reinhardtii (Table 3). These results are qualitatively consistent with the notion that hydrocarbon accumulation in microalgae is accompanied with a parallel accumulation of carotenoids (Eroglu and Melis, supra, 2010).

TABLE 3
Spectrophotometric determination of chlorophyll and total
carotenoid content in various Botryococcus species and
Chlamydomonas reinhardtii strain CC503.

| | | Chl | | |
|---|---|---|---|---|
| | Total Chl, | a/Chl b | Total Car, | Car/Chl |
| Strain | (% of dw) | (mol:mol) | (% of dw) | (w:w) |
| B. braunii var. Showa | 0.49 | 2.2 | 0.21 | 0.43 |
| B. braunii (Kawaguchi-1) | 0.86 | 2.3 | 0.54 | 0.63 |
| B. braunii (Yamanaka) | 0.39 | 2.0 | 0.10 | 0.26 |
| B. braunii (UTEX 2441) | 0.38 | 2.5 | 0.19 | 0.50 |
| B. braunii (UTEX LB572) | 0.64 | 2.1 | 0.13 | 0.20 |
| B. sudeticus (UTEX 2629) | 1.60 | 2.9 | 0.42 | 0.26 |
| Chlamydomonas | 2.05 | 2.2 | 0.37 | 0.18 |
| reinhardtii (CC503) | | | | |

Total lipophilic extracts in methanol were evaporated to dryness and the dry product was measured gravimetrically (Table 4, column 2). These extracts contained, in addition to any accumulated terpenoid or alkadiene hydrocarbons, membrane lipid diglycerides (DG) and photosynthetic pigments (Chl & Car). In green microalgae, most of the membrane lipid diglycerides and all pigments (Chl & Car) originate from the dominant thylakoid membranes, with relatively smaller DG contributions from the plasma membrane, endoplasmic reticulum, Golgi apparatus and mitochondria. On that basis, and given the similar Chl a/Chl b ratio among the strains examined, it was reasonable to assume a fairly similar total membrane DG lipid to Chl ratio among all microalgal strains in this study. Thus, the “membrane DG lipid” to Chl ratio parameter was employed as a normalization factor, and served to help us partition the “total lipophilic extract” of the strains into “membrane lipids” and “accumulated terpenoid or alkadiene hydrocarbons”, as follows (Table 4).

TABLE 4
Total amount of lipophilic extract, lipophilic extract to chlorophyll ratios,
and estimates of intracellular lipids and accumulated hydrocarbons
in various Botryococcus strains and Chlamydomonas reinhardtii.

| | | Total | | |
|---|---|---|---|---|
| | Total | lipophilic | | |
| | lipophilic | extract | Membrane | Accumulated |
| | extract | to Chl | lipids | hydrocarbons |
| Strain | (% of dw) | ratio, w/w | (% of dw) | (% of dw) |
| B. braunii var. | 33.91 | 69.2 | 5.01 | 28.9 |
| Showa | | | | |
| B. braunii var. | 28.37 | 33.0 | 8.97 | 19.4 |
| Kawaguchi-1 | | | | |
| B. braunii var. | 18.02 | 46.2 | 3.92 | 14.1 |
| Yamanaka | | | | |
| B. braunii var. | 16.71 | 44.0 | 3.91 | 12.8 |
| UTEX 2441 | | | | |
| B. braunii var. | 15.90 | 24.8 | 6.40 | 9.5 |
| UTEX LB572 | | | | |
| B. sudeticus var. | 19.32 | 12.0 | 16.12 | 3.2 |
| UTEX 2629 | | | | |
| C. reinhardtii var. | 20.46 | 10.0 | 20.46 | — |
| CC503 | | | | |

Chlamydomonas reinhardtii does not accumulate terpenoid or alkadiene hydrocarbon products (Eroglu and Melis, supra, 2009) and, consequently, has the lowest “total lipophilic extract” to Chl ratio (10.0:1) among the strains examined (Table 4, column 3). The “total lipophilic extract” in C. reinhardtii originates from membrane DG lipids and photosynthetic pigments in the cell. For the analysis below, we assumed that all strains examined have the same membrane DG lipid to Chl ratio (10.0:1), as in C. reinhardtii. This assumption was based on the similar Chl a/Chl b ratios measured in all strains (Table 3), suggesting that all strains have the same organization of thylakoid membranes, hence the same DG/Chl ratio. It follows that “total lipophilic extract” to Chl ratios greater than 10:1 would reflect the accumulated terpenoid or alkadiene hydrocarbons (Table 4).

Upon applying the C. reinhardtii “total lipophilic extract” to Chl ratio as the “membrane lipid” to Chl ratio in the other microalgae examined, we were able to estimate the membrane lipid content and the extra (accumulated) terpenoid or alkadiene hydrocarbons in the species examined. Results from such partitioning of the “total lipophilic extract” into “membrane lipids” and “accumulated hydrocarbons” are shown in Table 4 (columns 4 and 5). It was determined that Showa and Kawaguchi accumulated about 28.9% and 19.4% of their dw, respectively, in the form of extracellular hydrocarbons. The remaining A-race “braunii” strains accumulated 14.1-9.5% of their dw in the form of such hydrocarbons, whereas B. sudeticus had only baseline levels of extra (accumulated) hydrocarbons.

In greater detail, total lipophilic extract to Chl ratio for Showa (69.2:1) was much higher than that in C. reinhardtii (10.0:1), consistent with the notion of a relatively high extracellular botryococcene present in the former. Total lipophilic extract in Showa partitioned into 5.01% membrane lipids and 28.9% accumulated hydrocarbons. The total lipophilic extract to Chl ratio was intermediate for Kawaguchi (33.0:1), partitioning in 8.97% membrane lipids and 19.4% accumulated hydrocarbons. A-race strains Yamanaka, UTEX 2441, and UTEX LB572 had total lipophilic extract to Chl ratio in the 24.8-46.2:1 range, resulting in estimates of accumulated hydrocarbons in the 13-19% range (Table 4). Botryococcus sudeticus had a rather low total lipophilic extract to Chl ration (12.0:1) suggesting that this strain was poor in accumulated hydrocarbons. In summary, the higher “total lipophilic extract”/Chl ratio in the Botryococcus braunii strains reflects the accumulation of terpenoid or alkadiene hydrocarbon products. It may thus be concluded that all “braunii” strains synthesize and accumulate hydrocarbons above and beyond those that are encountered as membrane lipids, so as to attain “total lipophilic extract” to Chl ratio>10.0:1.

These gravimetric results are consistent with the density equilibrium (Table 2, 3rd column) and spectrophotometric (Table 2, 4th column) quantitation of hydrocarbons in the samples examined. The results are also consistent with measurements in the literature. For example, Wolf et al. (supra, 1985) reported that B. braunii var. Showa accumulates 24-29% of its dry biomass in the form botryococcene hydrocarbons. Yamaguchi et al. (supra, 1987) measured 34 g hydrocarbons per 100 g dw from the B. braunii Berkeley (Showa) strain. Nonomura (supra, 1988) reported greater botryococcene hydrocarbon content in Showa (30%, or more, per dry cell weight) than that in other strains of B. braunii (1.5 to 20%). Okada et al. (supra, 1995) also showed that B. braunii Kawaguchi-1 and Yamanaka micro-colonies accumulate hydrocarbons in the range of 18.8±0.8 and 16.1±0.3% of dry cell weight, respectively.

### Discussion

### Example 6

Green microalgae of the genus Botryococcus constitutively synthesize, accumulate, and secrete substantial amounts of their photosynthate as alkadiene (A-race microalgae) or tri-terpenoid (B-race microalgae) hydrocarbons. However, a direct quantitative analysis of the productivities by various Botryococci has been missing from the literature. For example, Sawayama et al. (supra, 1994) reported a biomass accumulation rate of only about 28 mg dw L⁻¹ d⁻¹ from the culture of Botryococcus braunii UTEX LB-572, grown in secondarily treated sewage in a continuous bioreactor system.

Also working with the UTEX LB-572 strain, grown in secondarily treated piggery wastewater in a batch reactor, An et al. (supra, 2003) reported biomass yield of ˜8.5 g dw per L and about 0.95 g hydrocarbon L⁻¹ after 12-day batch cultivation. On the other hand, upon growth in flasks under orbital shaking, Vazquez-Duhalt and Arredondo-Vega (Phytochemistry 30:2919-2925, 1991) reported biomass yield of about 300 mg dw L⁻¹ for both the B. braunii Austin and Gottingen strains (A-Race) following 28-day batch cultivation. Dayananda et al. (Process Biochemistry 40(9):3125-3131, 2005) cultivated B. braunii var. SAG 30.81 under diurnal (16 h light: 8 h dark) cycles in orbitally shaken conical flasks and reported a yield of 650 mg dw L⁻¹ after 30-day batch cultivation. It is evident from these results that B. braunii growth conditions, including bioreactor design and growth media composition, affect the productivity of the cultures. The present invention provides, for the first time, comparative hydrocarbon productivities in cultures of six different Botryococcus strains, grown under identical experimental conditions.

Multiple independent hydrocarbon quantitation methods on a variety of Botryococcus strains have not been applied before. Accordingly, Botryococcus productivity comparisons in the literature are based on sometimes substantially different quantitation methods. The present invention provides testing and validation of the applicability of three different and independent approaches and measurements for the quantitative measurement of hydrocarbons in various strains of the green microalgae Botryococcus. These methods were applied to six different strains of Botryococcus, belonging either to the A-race or B-race. Included were (i) density equilibrium of intact micro-colony measurements, (ii) spectrophotometric quantitation of extracellular hydrocarbons, and (iii) gravimetric measurements of the extracts. All three analytical methods yielded comparable quantitative results. Evidence revealed that the B-race microalgae Botryococcus braunii var. Showa and var. Kawaguchi-1 accumulated the highest amount of hydrocarbons per dry weight biomass, equivalent to about 30% (w:w) and 20% (w:w), respectively. The methods described herein will find important application in high throughput screening and selection of microalgae with substantial hydrocarbon productivity for commercial exploitation.

This example thus demonstrated that the methods of the invention for quantifying extracellular hydrocarbons are comparable to other methods and thus provide a surprisingly effective, efficient quantification method.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, accession numbers, patents, and patent applications cited in this specification are herein incorporated by reference as if each was specifically and individually indicated to be incorporated by reference.

1. A method of extracting extracellular botryococcene and methylated squalene terpenoid hydrocarbons from Botryococcus microalgae micro-colonies, the method comprising:
providing a sample comprising Botryococcus microalgae micro-colonies;
mechanically dispersing the microalgae micro-colonies, wherein the dispersal is performed without substantially breaking open the cells;
extracting the terpenoid hydrocarbons using an organic solvent selected from the group consisting of hexane, heptane or octane to obtain a fraction comprising the organic solvent containing the hydrocarbons;
quantifying the terpenoid hydrocarbons present in the organic solvent fraction spectrophotometrically

2. The method of claim 1, wherein the step of quantifying the terpenoid hydrocarbons present in the organic solvent spectrophotometrically comprises using an extinction coefficient of about 90±5 mM⁻¹ cm⁻¹ for the absorbance of the hydrocarbons at 190 nm.

3. The method of claim 1, wherein the microalgae is Botryococcus braunii.

4. The method of claim 3, wherein the Botryococcus braunii is Botryococcus braunii, var Showa.

5. The method of claim 1, wherein the organic solvent is heptane.

6. The method of claim 1, wherein the steps of mechanically dispersing the microalgae micro-colonies and extracting the terpenoid hydrocarbons is performed concurrently, and further, wherein the steps comprise vortexing the microalgae micro-colonies in the organic solvent in the presence of glass beads.

7. The method of claim 1, further comprising a step of heating the sample to about 100° C. prior to mechanically disrupting the micro-colonies.

8. The method of claim 1, wherein the step of mechanically disrupting the micro-colonies comprises sonicating the micro-colonies at low power.

9. A method of extracting extracellular botryococcenes and methylated squalenes from Botryococcus microalgae micro-colonies, the method comprising:
providing a sample comprising Botryococcus microalgae micro-colonies;
heating the sample to about 100° C. for 30 minutes or less;
vortexing the Botryococcus micro-colonies in heptane in the presence of glass beads to obtain a fraction comprising heptane containing the hydrocarbons; and
quantifying the botryococcene and methylated squalenes present in the organic solvent spectrophotometrically using an extinction coefficient of about 90±5 mM⁻¹ cm⁻¹ for the absorbance of the hydrocarbons at 190 nm.

10. The method of claim 9, wherein the Botryococcus sp. is Botryococcus braunii.

11. A method of extracting extracellular botryoxanthin from Botryococcus micro-colonies, the method comprising:
providing a sample comprising green algae micro-colonies;
vortexing the micro-colonies in heptane in the presence of glass beads to obtain a fraction comprising heptane containing the hydrocarbons;
quantifying the botryoxanthin present in the heptane fraction spectrophotometrically at 450 nm using an extinction coefficient of about 165±5 mM⁻¹ cm⁻¹.

12. The method of claim 11, wherein the microalgae is a Botryococcus braunii.

13. The method of claim 12, wherein the Botryococcus braunii is a member of the B race of Botryococcus.

14. A method of obtaining extracellular botryococcenes and methylated squalenes terpenoid hydrocarbons from Botryococcus microalgae micro-colonies, the method comprising:
providing a sample comprising Botryococcus microalgae micro-colonies;
heating the sample to about 100° C. for 30 minutes or less;
mechanically dispersing the Botryococcus micro-colonies in an aqueous medium to obtain an aqueous suspension comprising the unbroken cells and released terpenoid hydrocarbons;
separating the terpenoid hydrocarbons from the medium;
solubilizing the terpenoid hydrocarbons in heptane, hexane, or octane; and
quantifying the botryococcene hydrocarbons spectrophotometrically using an extinction coefficient of about 90±5 mM⁻¹ cm⁻¹ for the absorbance of the hydrocarbons at 190 nm.

15. The method of claim 14, wherein the step of separating the terpenoid hydrocarbons from the medium comprises allowing the terpenoid hydrocarbons to float to the top of the aqueous suspension.

16. The method of claim 14, wherein the step of separating the terpenoid hydrocarbons from the medium comprises centrifugation of the aqueous suspension.

17. A method of obtaining extracellular botryoxanthin from Botryococcus micro-colonies, the method comprising:
providing a sample comprising Botryococcus microalgae micro-colonies;
heating the sample to about 100° C. for 30 minutes or less;
mechanically dispersing the Botryococcus micro-colonies in an aqueous medium to obtain an aqueous suspension comprising the unbroken cells and released botryoxanthin;
separating the botryoxanthin from the medium; and
quantifying the botryoxanthin spectrophotometrically at 450 nm using an extinction coefficient of about 165±5 mM⁻¹ cm⁻¹.

18. The method of claim 17, wherein the step of separating the terpenoid hydrocarbons from the medium comprises allowing the terpenoid hydrocarbons to float to the top of the aqueous suspension.

19. The method of claim 17, wherein the step of separating the terpenoid hydrocarbons from the medium comprises centrifugation of the aqueous suspension.
